(19) United States
(12) Patent Application Publication
(10) Pub. No.: US 2022/0304906 A1
(43) Pub. Date: Sep. 29, 2022

(54) METHOD FOR THE DECOLORIZATION OF KERATIN MATERIAL THAT HAS BEEN DYED USING AN ORGANOSILICON COMPOUND AND A PIGMENT

(71) Applicant: Henkel AG & Co. KGaA, Duesseldorf (DE)

(72) Inventors: Torsten LECHNER, Langenfeld (DE); Carsten MATHIASZYK, Essen (DE); Phillip JAISER, Langenfeld (DE)

(73) Assignee: Henkel AG & Co. KGaA, Duesseldorf (DE)

(21) Appl. No.: 17/635,342

(22) PCT Filed: Jun. 9, 2020
(86) PCT No.: PCT/EP2020/065913

§ 371 Date: Feb. 14, 2022

(30) Foreign Application Priority Data
Aug. 14, 2019 (DE) 10 2019 212 170.5

(51) Int. Cl. A61K 8/25; A61Q 5/08; A61K 8/41; A61K 8/891; A61K 8/34

(52) U.S. Cl. CPC A61K 8/25; A61Q 5/08; A61K 8/41; A61K 8/891; A61K 8/345; A61K 2800/884

(57) ABSTRACT

The objective of the present disclosure is a process for decolorizing keratin material which has been colored by the application of at least one organosilicon compound and at least one pigment, wherein a decolorizing agent comprising
(a) at least one solvent and
(b) at least one alkalizing agent
is applied to the dyed keratin material and rinsed off again after a contact time.

### CROSS-REFERENCE TO RELATED APPLICATION

This application is a U.S. National-Stage entry under 35 U.S.C. § 371 based on International Application No. PCT/EP2020/065913, filed Jun. 9, 2020, which was published under PCT Article 21(2) and which claims priority to German Application No. 102019212170.5, filed Aug. 14, 2019, which are all hereby incorporated in their entirety by reference.

### TECHNICAL FIELD

The present application is in the field of cosmetics and relates to a process for decolorizing keratin material that has been colored by applying at least one organosilicon compound and at least one pigment. The decolorizing agent applied in this process is exemplified by its content of at least one solvent and at least one alkalizing agent. The decolorizing agent is applied to the dyed keratin material and rinsed off again after a reaction time.

A second object of the present application is a process for dyeing and later decolorizing keratin material, in which first a dyeing agent comprising an organosilicon compound and a pigment is applied to the keratin, and later the decolorization is conducted by applying the decolorizing agent described above.

A third object of the present application is a multi-component packaging unit which comprises the coloring agent and the decolorizing agent in separately prepared containers. Finally, a fourth object is the use of the decolorizing agent to decolorize appropriately colored keratin material.

### BACKGROUND

The change in shape and color of keratin fibers, especially hair, is a key area of modern cosmetics. To change the hair color, the expert knows various coloring systems depending on coloring requirements. Oxidation dyes are usually used for permanent, intensive dyeings with good fastness properties and good grey coverage. Such dyes usually contain oxidation dye precursors, so-called developer components and coupler components, which form the actual dyes with one another under the influence of oxidizing agents, such as hydrogen peroxide. Oxidation dyes are exemplified by very long-lasting dyeing results.

When direct dyes are used, ready-made dyes diffuse from the colorant into the hair fiber. Compared to oxidative hair dyeing, the dyeings obtained with direct dyes have a shorter shelf life and quicker wash ability. Dyeing with direct dyes usually remain on the hair for a period of between 5 and 20 washes.

The use of color pigments is known for short-term color changes on the hair and/or skin. Color pigments are understood to be insoluble, coloring substances. These are present undissolved in the dye formulation in the form of small particles and are only deposited from the outside on the hair fibers and/or the skin surface. Therefore, they can usually be removed again without residue by a few washes with detergents comprising surfactants. Various products of this type are available on the market under the name hair mascara.

If the user wants particularly long-lasting dyeings, the use of oxidative dyes has so far been his only option. However, despite numerous optimization attempts, an unpleasant ammonia or amine odor cannot be completely avoided in oxidative hair dyeing. The hair damage still associated with the use of oxidative dyes also has a negative effect on the user's hair.

Recently, a new dyeing system has received attention in which dyeings are colored by applying a combination of a pigment, an organic silicon compound, and a polymer. This new dyeing system is described, for example, in EP 2168633 B1.

When these organic silicon compounds are applied to keratinous material, a film or coating is formed on the keratinous material, which completely envelops the keratinous material and, in this way, strongly influences the properties of the keratinous material. If the application is carried out in the presence of a colorant compound, for example a pigment, the pigments are embedded in this film or coating. The film colored by the pigment remains on the keratin material or keratin fibers. The colorations obtained in this way are said to be particularly resistant to shampooing.

Even with this dyeing process, it may happen that the dyeing is to be partially or completely reversed for several reasons. Partial removal of the dyeing may be necessary, for example, if the dyeing result on the fibers turns out darker than desired. On the other hand, complete removal of the stain may also be desired in some cases. For example, it is conceivable that the hair should be dyed or tinted in a certain shade for a specific occasion, and after a few days the original color should be recovered.

### BRIEF SUMMARY

Processes of decoloring keratinous material and multi-component packing units are provided herein. In an embodiment, a process is provided for decolorizing keratinous material which has been colored by the application of at least one organosilicon compound and at least one pigment, wherein the process comprises:

applying a decolorizing agent to the keratinous material, wherein the decolorizing agent comprises:

(a) at least one solvent, and

(b) at least one alkalizing agent; and

rinsing the decolorizing agent from the keratinous material after a contact time.

In another embodiment, a multi-component packaging unit is provided for staining and decolorizing keratin material. The multi-component packaging unity includes separately packaged:

a first container with an agent comprising one or more organic C₁-C₆ alkoxy silanes and/or their condensation products,

a second container with an agent comprising at least one pigment,

a third container with a decolorizing agent as defined in claim 1, and

optionally a fourth container with an agent comprising at least one film-forming polymer.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description. It is to be appreciated that all values as provided herein, save for the actual examples, are approximate values with endpoints or particular values intended to be read as “about” or “approximately” the value as recited.

The task of the present disclosure was therefore to provide a decolorizing agent for decolorizing dyed keratinous fibers, which have previously been dyed by the application of at least one organosilicon compound, an alkoxy silane, and at least one pigment. Here, the decolorization should be as complete as possible, so that the coloration of the keratin material can ideally be restored to its original state. Furthermore, the decolorization should be long-lasting and uniform, and the decolorized keratin fibers should suffer neither shifts in nuance nor irregularities in the color result. In addition, the keratin material should be damaged as little as possible by the decolorizing agent.

Surprisingly, it has now been found that this task can be fully solved if keratinous material previously colored with at least one organosilicon compound and with at least one pigment is treated with a decolorizing agent comprising at least one solvent and at least one alkalizing agent.

A first object of the present disclosure is a method for decolorizing keratin material which has been colored by application of at least one organosilicon compound and at least one pigment, wherein a decolorizing agent comprising

(a) at least one solvent and

(b) at least one alkalizing agent

is applied to the dyed keratin material and rinsed off again after a contact time.

For example, in the experiments leading to the present disclosure, keratin fibers (hair strands) were used which had previously been dyed with an agent comprising various organosilicon compounds (such as alkoxysilanes) and various pigments (such as organic and inorganic pigments). After being dyed, these fibers were decolorized again by applying a decolorizing agent as contemplated herein. In this context, it has been surprisingly found that a decolorizing agent comprising the combination of solvent (a) and alkalizing agent (b) is capable of completely removing the colored film formed from organosilicon compound and pigment from the keratin fiber. In this way it became possible to restore the original coloration of the hair and return the keratin fibers to their original color state.

### Decolorization of Keratinous Material

Keratinous material includes hair, skin, nails (such as fingernails and/or toenails). Wool, furs and feathers also fall under the definition of keratinous material. Preferably, keratinous material is understood to be human hair, human skin and human nails, especially fingernails and toenails. Keratinous material is understood to be human hair.

In the context of the present disclosure, the term “decolorizing agent” is understood to mean that a coloration produced by the application of at least one organosilicon compound and at least one pigment can be removed again on the keratin material. In this dyeing process, the keratin material or keratin fiber is coated with a colored film formed from the organosilicon compounds and pigments. As contemplated herein, the application of the decolorizing agent takes place after the application of the colorant and causes the removal of this colored film from the keratin material.

Characteristic of the process as contemplated herein is the application of the decolorizing agent to keratin material previously colored by application of at least one organosilicon compound and at least one pigment.

Coloring with the Use of Organosilicon Compounds

In the process as contemplated herein, the decolorizing agent is applied to previously colored keratin material. In the previous dyeing process, at least one organosilicon compound is used on the keratin material.

Organic silicon compounds, alternatively called organosilicon compounds, are compounds which either have a direct silicon-carbon bond (Si—C) or in which the carbon is bonded to the silicon atom via an oxygen, nitrogen or sulfur atom. The organic silicon compounds of the present disclosure are preferably compounds comprising one to three silicon atoms. Organic silicon compounds preferably comprise one or two silicon atoms.

The decolorizing agent works particularly well on dyed keratin material if an organic C₁-C₆ alkoxy silane was used in the previous dyeing.

The organic C₁-C₆ alkoxy silane(s) are organic, non-polymeric silicon compounds, preferably selected from the group of silanes comprising one, two or three silicon atoms.

According to IUPAC rules, the term silane stands for a group of chemical compounds based on a silicon skeleton and hydrogen. In organic silanes, the hydrogen atoms are completely or partially replaced by organic groups such as (substituted) alkyl groups and/or alkoxy groups.

A characteristic feature of the C₁-C₆ alkoxy silanes of the present disclosure is that at least one C₁-C₆ alkoxy group is directly bonded to a silicon atom. The C₁-C₆ alkoxy silanes as contemplated herein thus comprise at least one structural unit R′R″R′″Si—O—(C₁-C₆ alkyl) where the radicals R′, R″ and R′″ stand for the three remaining bond valencies of the silicon atom.

The C₁-C₆ alkoxy group or groups bonded to the silicon atom are very reactive and are hydrolyzed at high rates in the presence of water, the reaction rate depending, among other things, on the number of hydrolysable groups per molecule. If the hydrolysable C₁-C₆ alkoxy group is an ethoxy group, the organic silicon compound preferably comprises a structural unit R′R″R′″Si—O—CH2-CH3. The R′, R″ and R′″ residues again represent the three remaining free valences of the silicon atom.

Even the addition of insignificant amounts of water leads first to hydrolysis and then to a condensation reaction between the organic alkoxy silanes. For this reason, both the organic alkoxy silanes and their condensation products may be present in the coloring composition.

A condensation product is understood to be a product formed by reaction of at least two organic C₁-C₆ alkoxy silanes with elimination of water and/or with elimination of a C₁-C₆ alkanol.

The condensation products can, for example, be dimers, or even trimers or oligomers, where in the condensation products are always in balance with the monomers.

Depending on the amount of water used or consumed in the hydrolysis, the equilibrium shifts from monomeric C₁-C₆ alkoxysilane to condensation product.

In a very particularly preferred embodiment, the decolorizing agent is applied to keratin material which has been colored by application of at least one organic C₁-C₆ alkoxy-silane and/or a condensation product thereof and by application of at least one pigment.

The organic C₁-C₆ alkoxy-silanes may be, for example, compounds selected from silanes having one, two or three silicon atoms, the organic silicon compound further comprising one or more basic chemical functions.

This basic group can be, for example, an amino group, an alkylamino group or a dialkylamino group, which is preferably connected to a silicon atom via a linker. Preferably, the basic group is an amino group, a C₁-C₆ alkylamino group or a di(C₁-C₆)alkylamino group.

In a very particularly preferred process as contemplated herein the decolorizing agent is applied to keratin material which has been colored by application of at least one organic C₁-C₆ alkoxy silane and/or a condensation product thereof and by application of at least one pigment, wherein the C₁-C₆ alkoxy silane further comprises one or more basic chemical functions.

Particularly satisfactory results were obtained when the decolorizing agent was applied to keratin material previously dyed with at least one C₁-C₆ alkoxy silane of formula (S-I) and/or (S-II). Since, as previously described, hydrolysis/condensation already starts at traces of moisture, the coloring of the keratin material with C₁-C₆ alkoxy silanes of the formula (S-I) and/or (S-II) also the use of their condensation products.

In another very particularly preferred embodiment, the decolorizing agent is applied to keratin material which has been colored by application of at least one organic C₁-C₆ alkoxy-silane of the formula (S-I) and/or (S-II) and a pigment,

R₁R₂N-L-Si(OR₃)ₐ(R₄)_{b} (S-I)

where
R₁, R₂ independently represent a hydrogen atom or a C₁-C₆ alkyl group,
L is a linear or branched divalent C₁-C₂₀ alkylene group,
R3, R4 independently of one another represent a C₁-C₆ alkyl group,
a, stands for an integer from 1 to 3, and
b stands for the integer 3-a, and

(R₅O)_{c}(R₆)_{d}Si-(A)ₑ-[NR₇-(A′)]_{f}—[O-(A″)]_{g}—[NR₈-(A′″)]ₕ—Si(R₆′)_{d′}(OR₅′)_{c′} (S-II),

where
R₅, R₅′, R₅″, R₆, R₆′ and R₆″ independently represent a C₁-C₆ alkyl group,
A, A′, A″, A′″ and A″″ independently represent a linear or branched divalent C₁-C₂₀ alkylene group,
R₇ and R₈ independently represent a hydrogen atom, a C₁-C₆ alkyl group, a hydroxy C₁-C₆ alkyl group, a C₂-C₆ alkenyl group, an amino C₁-C₆ alkyl group or a group of formula (S-III),

-(A″″)-Si(R₆″)_{d}″(OR₅″)_{c}″ (S-III),
c, stands for an integer from 1 to 3,
d stands for the integer 3-c,
c′ stands for an integer from 1 to 3,
d′ stands for the integer 3-c′,
c″ stands for an integer from 1 to 3,
d″ stands for the integer 3-c″,
e stands for 0 or 1,
f stands for 0 or 1,
g stands for 0 or 1,
h stands for 0 or 1,
provided that at least one of e, f, g and h is different from 0.

The substituents R₁, R₂, R₃, R₄, R₅, R₅′, R₅″, R₆, R₆′, R₆″, R₇, R₈, L, A, A′, A″, A′″ and A″″ in the compounds of formula (S-I) and (S-II) are explained below as examples: Examples of a C₁-C₆ alkyl group are the groups methyl, ethyl, propyl, isopropyl, n-butyl, s-butyl and t-butyl, n-pentyl and n-hexyl. Propyl, ethyl and methyl are preferred alkyl radicals. Examples of a C₂-C₆ alkenyl group are vinyl, allyl, but-2-enyl, but-3-enyl and isobutenyl, preferred C₂-C₆ alkenyl radicals are vinyl and allyl. Preferred examples of a hydroxy C₁-C₆ alkyl group are a hydroxymethyl, a 2-hydroxyethyl, a 2-hydroxypropyl, a 3-hydroxypropyl, a 4-hydroxybutyl group, a 5-hydroxypentyl and a 6-hydroxyhexyl group; a 2-hydroxyethyl group is particularly preferred. Examples of an amino C₁-C₆ alkyl group are the aminomethyl group, the 2-aminoethyl group, the 3-aminopropyl group. The 2-aminoethyl group is particularly preferred. Examples of a linear bivalent C₁-C₂₀ alkylene group include the methylene group (—CH₂—), the ethylene group (—CH₂—CH₂—), the propylene group (—CH₂—CH₂—CH₂—), and the butylene group (—CH₂—CH₂—CH₂—CH₂—). The propylene group (—CH₂—CH₂—CH₂—) is particularly preferred. From a chain length of 3 C atoms, bivalent alkylene groups can also be branched. Examples of branched divalent, bivalent C₃-C₂₀ alkylene groups are (—CH₂—CH(CH₃)—) and (—CH₂—CH(CH₃)—CH₂—).

In the organic silicon compounds of the formula (S-I)

R₁R₂N-L-Si(OR₃)ₐ(R₄)_{b} (S-I),

the radicals R₁ and R₂ independently of one another represent a hydrogen atom or a C₁-C₆ alkyl group. Very preferably, R₁ and R₂ both represent a hydrogen atom.

In the middle part of the organic silicon compound is the structural unit or the linker -L- which stands for a linear or branched, divalent C₁-C₂₀ alkylene group. The divalent C₁-C₂₀ alkylene group may alternatively be referred to as a divalent or divalent C₁-C₂₀ alkylene group, by which is meant that each—L grouping may form—two bonds.

Preferably -L- stands for a linear, bivalent C₁-C₂₀ alkylene group. Further preferably -L- stands for a linear bivalent C₁-C₆ alkylene group. Particularly preferred -L stands for a methylene group (CH₂—), an ethylene group (—CH₂—CH₂—), propylene group (—CH₂—CH₂—CH₂—) or butylene (—CH₂—CH₂—CH₂—CH₂—). L stands for a propylene group (—CH₂—CH₂—CH₂—)

The organic silicon compounds of formula (S-I) as contemplated herein.

R₁R₂N-L-Si(OR₃)ₐ(R₄)_{b} (S-I),

one end of each carries the silicon-comprising group —Si(OR₃)ₐ(R₄)_{b}.

In the terminal structural unit —Si(OR₃)ₐ(R₄)_{b} R3 and R4 independently represent a C₁-C₆ alkyl group, and particularly preferably R₃ and R₄ independently represent a methyl group or an ethyl group.

Here a stands for an integer from 1 to 3, and b stands for the integer 3-a. If a stands for the number 3, then b is equal to 0. If a stands for the number 2, then b is equal to 1. If a stands for the number 1, then b is equal to 2.

The application of the decolorizing agent as contemplated herein was particularly successful if the keratin material had previously been colored with an organic C₁-C₆ alkoxy silane of the formula (S-I) in which the radicals R₃, R₄ independently of one another represent a methyl group or an ethyl group.

Furthermore, the application of the decolorizing agent as contemplated herein was also particularly successful if the keratin material had previously been dyed with an organic C₁-C₆ alkoxy silane of the formula (S-I) in which the radical a represents the number 3. In this case the radical b stands for the number 0.

In a further preferred embodiment, the decolorizing agent is applied to keratin material which has been colored by application of at least one organic C₁-C₆ alkoxy-silane of the formula (I) and/or a condensation product thereof and by application of at least one pigment,

R₁R₂N-L-Si(OR₃)ₐ(R₄)_{b} (S-I),

where
R₁, R₂ both represent a hydrogen atom, and
L represents a linear, bivalent C₁-C₆-alkylene group, preferably a propylene group (—CH₂—CH₂—CH₂—) or an ethylene group (—CH₂—CH₂—),
R₃ represents an ethyl group or a methyl group,
R₄ represents a methyl group or an ethyl group,
a stands for the number 3 and
b stands for the number 0.

Silica compounds of formula (I) which are particularly well removable by subsequent application of the decolorizing agent are

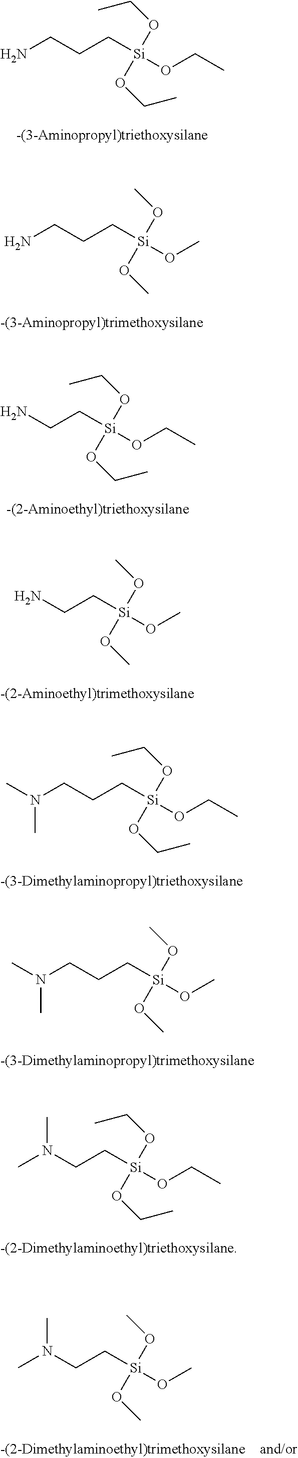

In a further preferred embodiment, the decolorizing agent is applied to keratin material which has been colored by application of at least one organic C₁-C₆ alkoxy-silane and/or a condensation product thereof and by application of at least one pigment, wherein the organic C₁-C₆ alkoxy-silane is selected from the group of
(3-Aminopropyl)triethoxysilane
(3-Aminopropyl)trimethoxysilane
(2-Aminoethyl)triethoxysilane
(2-Aminoethyl)trimethoxysilane
(3-Dimethylaminopropyl)triethoxysilane
(3-Dimethylaminopropyl)trimethoxysilane
(2-Dimethylaminoethyl)triethoxysilane and/or
(2-Dimethylaminoethyl)trimethoxysilane.

The organic silicon compound of formula (I) is commercially available. (3-aminopropyl)trimethoxysilane, for example, can be purchased from Sigma-Aldrich. Also (3-aminopropyl)triethoxysilane is commercially available from Sigma-Aldrich.

In a further embodiment of the process as contemplated herein, the keratin material can also be colored beforehand by applying one or more organic C₁-C₆ alkoxy silanes of the formula (S-II),

(R₅O)_{c}(R₆)_{d}Si-(A)ₑ-[NR₇-(A′)]_{f}—[O-(A″)]_{g}—[NR₈-(A′″)]ₕ—Si(R₆′)_{d′}(OR₅′)_{c′} (S-II).

The organosilicon compounds of the formula (S-II) as contemplated herein each carry at their two ends the silicon-comprising groupings (R₅O)_{c}(R₆)_{d}Si— and —Si(R₆′)_{d′}(OR₅′)_{c′}.

In the central part of the molecule of formula (S-II) there are the groups -(A)ₑ- and —[NR₇-(A′)]_{f}- and —[O-(A″)]_{g}- and —[NR₈-(A′″)]ₕ-. Here, each of the radicals e, f, g and h can independently of one another stand for the number 0 or 1, with the proviso that at least one of the radicals e, f, g and h is different from 0. In other words, an organic silicon compound of formula (II) as contemplated herein comprises at least one grouping from the group comprising -(A)- and —[NR₇-(A′)]- and —[O-(A″)]- and —[NR₈-(A′″)]-.

In the two terminal structural units (R₅O)_{c}(R₆)_{d}Si— and —Si(R₆′)_{d′}(OR₅′)_{c′}, the residues R5, R5′, R5″ independently represent a C₁-C₆ alkyl group. The radicals R6, R6′ and R6″ independently represent a C₁-C₆ alkyl group.

Here a stands for an integer from 1 to 3, and d stands for the integer 3-c. If c stands for the number 3, then d is equal to 0. If c stands for the number 2, then d is equal to 1. If c stands for the number 1, then d is equal to 2.

Analogously c′ stands for a whole number from 1 to 3, and d′ stands for the whole number 3-c′. If c′ stands for the number 3, then d′ is 0. If c′ stands for the number 2, then d′ is 1. If c′ stands for the number 1, then d′ is 2.

Dyeings with the best wash fastness values could be obtained if the residues c and c′ both stand for the number 3. In this case d and d′ both stand for the number 0.

In a further preferred embodiment, the decolorizing agent is applied to keratin material which has been colored by application of at least one organic C₁-C₆ alkoxy-silane of the formula (II) and/or a condensation product thereof and by application of at least one pigment,

(R₅O)_{c}(R₆)_{d}Si-(A)ₑ-[NR₇-(A′)]_{f}—[O-(A″)]_{g}—[NR₈-(A′″)]ₕ—Si(R₆′)_{d′}(OR₅′)_{c′} (S-II),

where
R₅ and R₅′ independently represent a methyl group or an ethyl group,
c and c′ both stand for the number 3 and
d and d′ both stand for the number 0.

When c and c′ are both 3 and d and d′ are both 0, the organic silicon compounds as contemplated herein correspond to the formula (S-IIa)

(R₅O)₃Si-(A)ₑ-[NR₇-(A′)]_{f}—[O-(A″)]_{g}—[NR₈-(A′″)]ₕ—Si(OR₅′)₃ (S-IIa).

The radicals e, f, g and h can independently stand for the number 0 or 1, whereby at least one radical from e, f, g and h is different from zero. The abbreviations e, f, g and h thus define which of the groupings -(A)ₑ- and —[NR₇-(A′)]_{f}- and —[O-(A″)]_{g}- and —[NR₈-(A′″)]ₕ- are in the middle part of the organic silicon compound of formula (II).

In this context, the presence of certain groupings has proved particularly beneficial. Particularly satisfactory results could be obtained if at least two of the residues e, f, g and h stand for the number 1. Especially preferred e and f both stand for the number 1. Furthermore, g and h both stand for the number 0.

When e and f are both 1 and g and h are both 0, the organic silicon compounds as contemplated herein are represented by the formula (S-IIb)

(R₅O)_{c}(R₆)_{d}Si-(A)-[NR₇-(A′)]—Si(R₆′)_{d′}(OR₅′)_{c′} (S-IIb).

The radicals A, A′, A″, A′″ and A″″ independently represent a linear or divalent, bivalent C₁-C₂₀ alkylene group. Preferably the radicals A, A′, A″, A′″ and A″″ independently of one another represent a linear, bivalent C₁-C₂₀ alkylene group. Further preferably the radicals A, A′, A″, A′″ and A″″ independently represent a linear bivalent C₁-C₆ alkylene group.

The divalent C₁-C₂₀ alkylene group may alternatively be referred to as a divalent or divalent C₁-C₂₀ alkylene group, by which is meant that each grouping A, A′, A″, A′″ and A″″ may form two bonds.

In particular, the radicals A, A′, A″, A′″ and A″″ independently of one another represent a methylene group (—CH₂—), an ethylene group (—CH₂—CH₂—), a propylene group (—CH₂—CH₂—CH₂—) or a butylene group (—CH₂—CH₂—CH₂—CH₂—). Very preferably, the radicals A, A′, A″, A′″ and A″″ represent a propylene group (—CH₂—CH₂—CH₂—).

If the radical f represents the number 1, then the organic silicon compound of formula (II) as contemplated herein comprises a structural grouping —[NR₇-(A′)]-. If the radical f represents the number 1, then the organic silicon compound of formula (II) as contemplated herein comprises a structural grouping —[NR₈-(A′″)]-.

Wherein R₇ and R₈ independently represent a hydrogen atom, a C₁-C₆ alkyl group, a hydroxy-C₁-C₆ alkyl group, a C₂-C₆ alkenyl group, an amino-C₁-C₆ alkyl group or a group of the formula (S-III)

(A″″)-Si(R₆″)_{d″}(OR₅″)_{c″} (S-III).

Very preferably the radicals R₇ and R₈ independently of one another represent a hydrogen atom, a methyl group, a 2-hydroxyethyl group, a 2-alkenyl group, a 2-aminoethyl group or a grouping of the formula (S-III).

If the radical f represents the number 1 and the radical h represents the number 0, the organic silicon compound as contemplated herein comprises the grouping [NR₇-(A′)] but not the grouping —[NR₈-(A′″)]. If the radical R₇ now stands for a grouping of the formula (III), the organic silicone compound comprises 3 reactive silane groups.

In a further preferred embodiment, the decolorizing agent is applied to keratin material which has been colored by application of at least one organic C₁-C₆ alkoxy-silane of the formula (II) and/or a condensation product thereof and by application of at least one pigment,

(R₅O)_{c}(R₆)_{d}Si-(A)ₑ-[NR₇-(A′)]_{f}—[O-(A″)]_{g}—[NR₈-(A′″)]ₕ—Si(R₆′)_{d′}(OR₅′)_{c′} (II),

where
e and f both stand for the number 1,
g and h both stand for the number 0,
A and A′ independently represent a linear, divalent C₁-C₆ alkylene group
   and
R7 represents a hydrogen atom, a methyl group, a 2-hydroxyethyl group, a 2-alkenyl group, a 2-aminoethyl group or a group of formula (S-III).

In a further preferred embodiment, the decolorizing agent is applied to keratin material which has been colored by application of at least one organic C₁-C₆ alkoxy-silane of the formula (I) and/or a condensation product thereof and by application of at least one pigment, where
e and f both stand for the number 1,
g and h both stand for the number 0,
A and A′ independently of one another represent a methylene group (—CH₂—), an ethylene group (—CH₂—CH₂—) or a propylene group (—CH₂—CH₂—CH₂),
   and
R7 represents a hydrogen atom, a methyl group, a 2-hydroxyethyl group, a 2-alkenyl group, a 2-aminoethyl group or a group of formula (S-III).

Silica compounds of formula (II) which can be removed particularly well by subsequent application of the decolorizing agent are

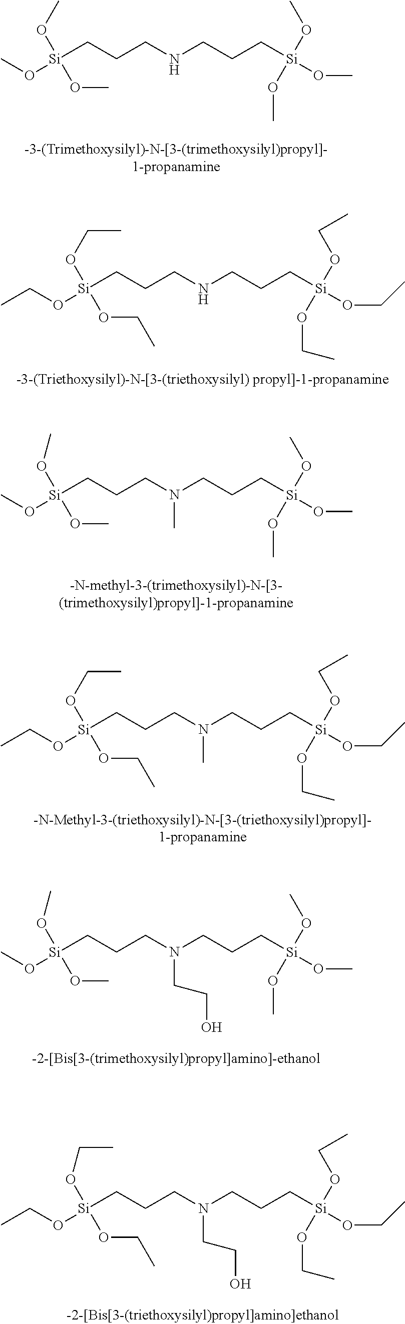

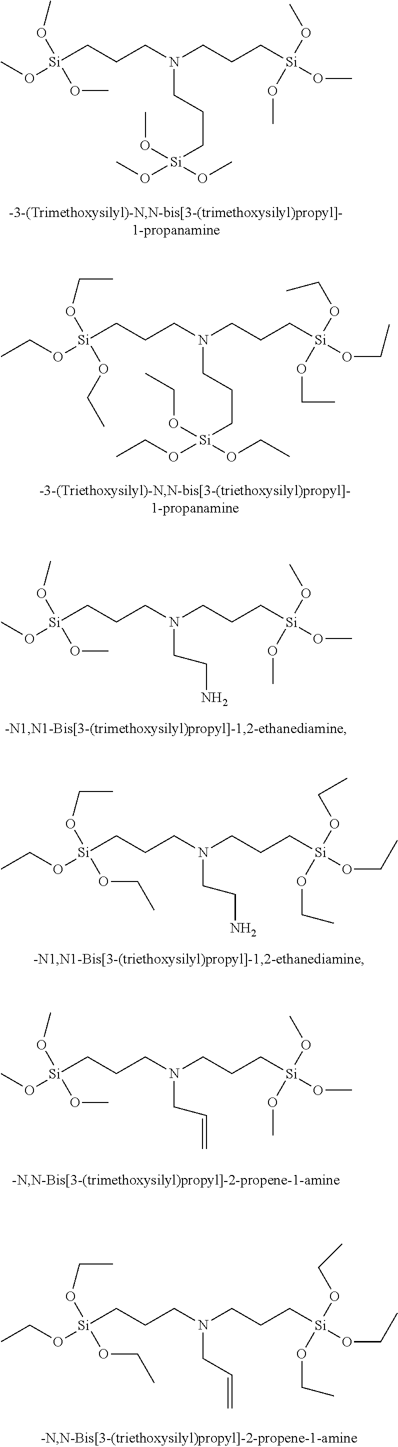

The organic silicon compounds of formula (S-II) are commercially available.

Bis(trimethoxysilylpropyl)amines with the CAS number 82985-35-1 can be purchased from Sigma-Aldrich.
Bis[3-(triethoxysilyl)propyl]amines with the CAS number 13497-18-2 can be purchased from Sigma-Aldrich, for example.
N-methyl-3-(trimethoxysilyl)-N-[3-(trimethoxysilyl)propyl]-1-propanamine is alternatively referred to as Bis(3-trimethoxysilylpropyl)-N-methylamine and can be purchased commercially from Sigma-Aldrich or Fluorochem.
3-(triethoxysilyl)-N,N-bis[3-(triethoxysilyl)propyl]-1-propanamine with the CAS number 18784-74-2 can be purchased for example from Fluorochem or Sigma-Aldrich.

In a further preferred embodiment, the decolorizing agent is applied to keratin material which has been colored by application of at least one organic C₁-C₆ alkoxy-silane and/or a condensation product thereof and by application of at least one pigment, wherein the organic C₁-C₆ alkoxy-silane is selected from the group of
3-(Trimethoxysilyl)-N-[3-(trimethoxysilyl)propyl]-1-propanamine
3-(Triethoxysilyl)-N-[3-(triethoxysilyl) propyl]-1-propanamine
N-Methyl-3-(trimethoxysilyl)-N-[3-(trimethoxysilyl)propyl]-1-propanamine
N-Methyl-3-(triethoxysilyl)-N-[3-(triethoxysilyl) propyl]-1-propanamine
2-[Bis[3-(trimethoxysilyl) propyl]amino]-ethanol
2-[Bis[3-(triethoxysilyl) propyl]amino]ethanol
3-(Trimethoxysilyl)-N,N-bis[3-(trimethoxysilyl) propyl]-1-propanamine
3-(Triethoxysilyl)-N,N-bis[3-(triethoxysilyl) propyl]-1-propanamine
N1,N1-Bis[3-(trimethoxysilyl) propyl]-1,2-ethanediamine,
N1,N1-Bis[3-(triethoxysilyl) propyl]-1,2-ethanediamine,
N,N-Bis[3-(trimethoxysilyl)propyl]-2-Propen-1-amine and/or
N,N-Bis[3-(triethoxysilyl)propyl]-2-propen-1-amine.

In further dyeing-decoloring trials, it has also been found to be particularly advantageous if, in the process as contemplated herein, at least one organic C₁-C₆ alkoxy silane (A2) of the formula (S-IV) has been used for dyeing the keratin material

R₉Si(OR₁₀)ₖ(R₁₁)ₘ (S-IV).

The compounds of formula (S-IV) are organic silicon compounds selected from silanes having one, two or three silicon atoms, wherein the organic silicon compound comprises one or more hydrolysable groups per molecule.

The organic silicon compound(s) of formula (S-IV) may also be referred to as silanes of the alkyl-C₁-C₆-alkoxy-silane type,

R₉Si(OR₁₀)ₖ(R₁₁)ₘ (S-IV),

where
R₉ represents a C₁-C₁₂ alkyl group,
R₁₀ represents a C₁-C₆ alkyl group,
R₁₁ represents a C₁-C₆ alkyl group
k is an integer from 1 to 3, and
m stands for the integer 3-k.

In a further particularly preferred embodiment, the decolorizing agent is applied to keratin material which has been colored by application of at least one organic C₁-C₆ alkoxy-silane of the formula (S-IV) and/or a condensation product thereof and by application of at least one pigment,

R₉Si(OR₁₀)ₖ(R₁₁)ₘ (S-IV),

where
R₉ represents a C₁-C₁₂ alkyl group,
R₁₀ represents a C₁-C₆ alkyl group,
R₁₁ represents a C₁-C₆ alkyl group
k is an integer from 1 to 3, and
m stands for the integer 3-k.

In the organic C₁-C₆ alkoxy silanes of formula (S-IV), the R₉ radical represents a C₁-C₁₂ alkyl group. This C₁-C₁₂ alkyl group is saturated and can be linear or branched. Preferably, R₉ represents a linear C₁-C₈ alkyl group. Preferably R₉ stands for a methyl group, an ethyl group, an n-propyl group, an n-butyl group, an n-pentyl group, an n-hexyl group, an n-octyl group or an n-dodecyl group. Particularly preferred, R₉ stands for a methyl group, an ethyl group or an n-octyl group.

In the organic silicon compounds of formula (S-IV), the radical R₁₀ represents a C₁-C₆ alkyl group. Highly preferred R₁₀ stands for a methyl group or an ethyl group.

In the organic silicon compounds of formula (S-IV), the radical Rn represents a C₁-C₆ alkyl group. Particularly preferably, Rn represents a methyl group or an ethyl group.

Furthermore, k stands for a whole number from 1 to 3, and m stands for the whole number 3-k. If k stands for the number 3, then m is equal to 0. If k stands for the number 2, then m is equal to 1. If k stands for the number 1, then m is equal to 2.

Particularly good decolorization results were obtained when the keratin material was first dyed with an organic C₁-C₆ alkoxy silane of the formula (S-IV) in which the radical k stands for the number 3. In this case the radical m stands for the number 0.

Organic silicon compounds of the formula (S-IV) which are particularly suitable for solving the problem as contemplated herein are

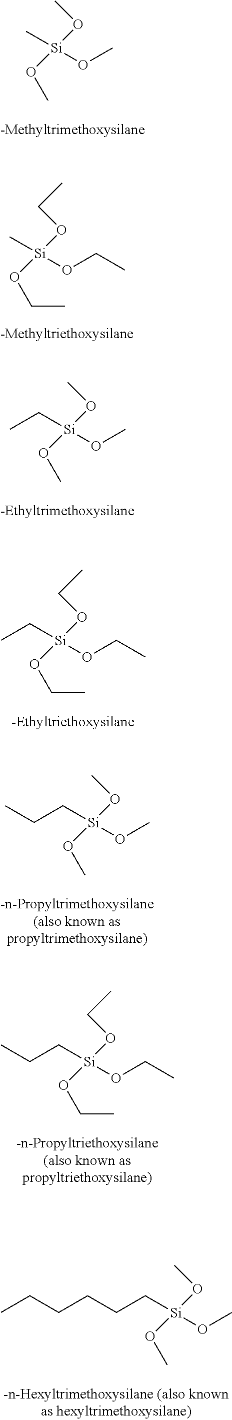

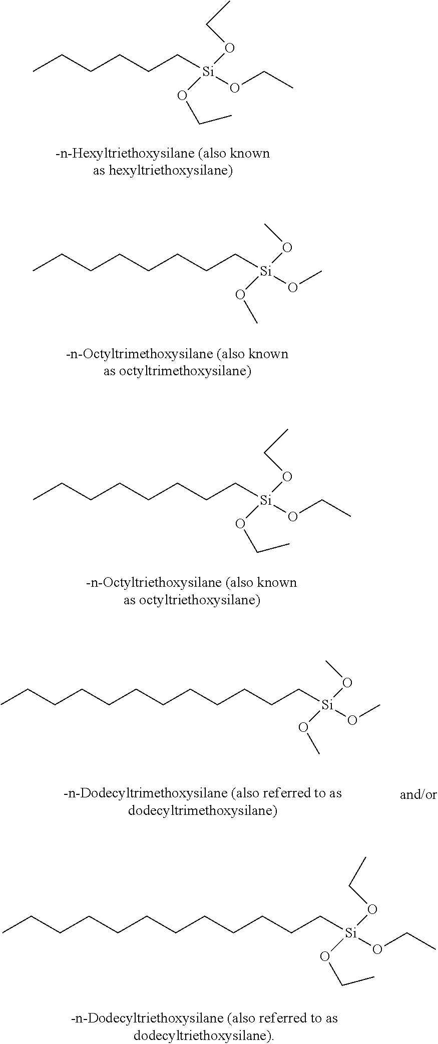

In a further preferred embodiment, the decolorizing agent is applied to keratin material which has been colored by application of at least one organic C₁-C₆ alkoxy-silane and/or a condensation product thereof and by application of at least one pigment, wherein the organic C₁-C₆ alkoxy-silane is selected from the group of
Methyltrimethoxysilane,
Methyltriethoxysilane,
Ethyltrimethoxysilane,
Ethyltriethoxysilane,
Propyltrimethoxysilane
Propyltrimethoxysilane
Hexyltrimethoxysilane,
Hexyltriethoxysilane,
Octyltrimethoxysilane,
Octyltriethoxysilane,
Dodecyltrimethoxysilane, and/or
Dodecyltriethoxysilane.

The corresponding hydrolysis or condensation products are, for example, the following compounds:

Hydrolysis of C₁-C₆ alkoxy silane of the formula (S-I) with water (reaction scheme using the example of 3-aminopropyltriethoxysilane):

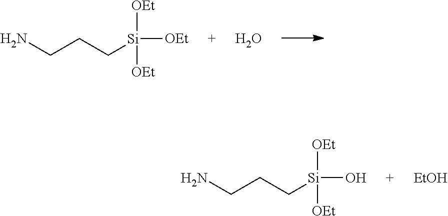 [SMILES: "EtOH" ; CCO[Si](CCCN)(OCC)OCC ; O ; CCO[Si](O)(CCCN)OCC]

Depending on the amount of water used, the hydrolysis reaction can also take place several times per C₁-C₆ alkoxy silane used:

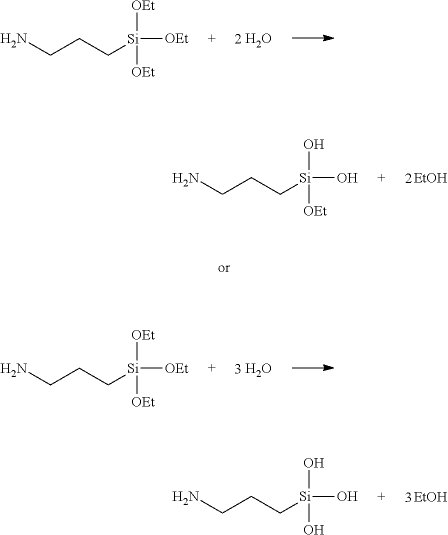

Hydrolysis of C₁-C₆ alkoxy silane of formula (S-IV) with water (reaction scheme using methyltrimethoxysilane as an example):

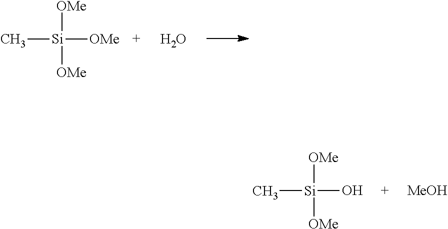

Depending on the amount of water used, the hydrolysis reaction can also take place several times per C₁-C₆ alkoxy silane used:

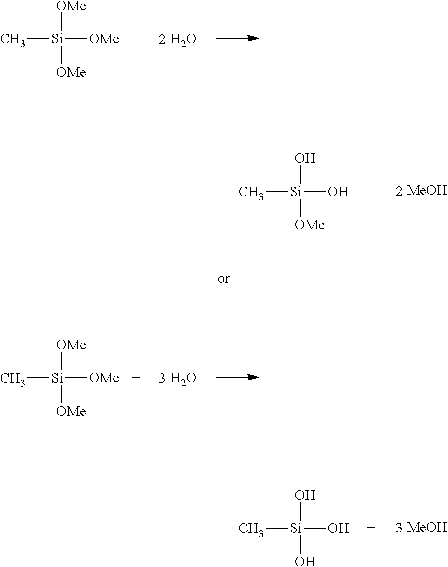

Condensation reactions include (shown using the mixture (3-aminopropyl)triethoxysilane and methyltrimethoxysilane):

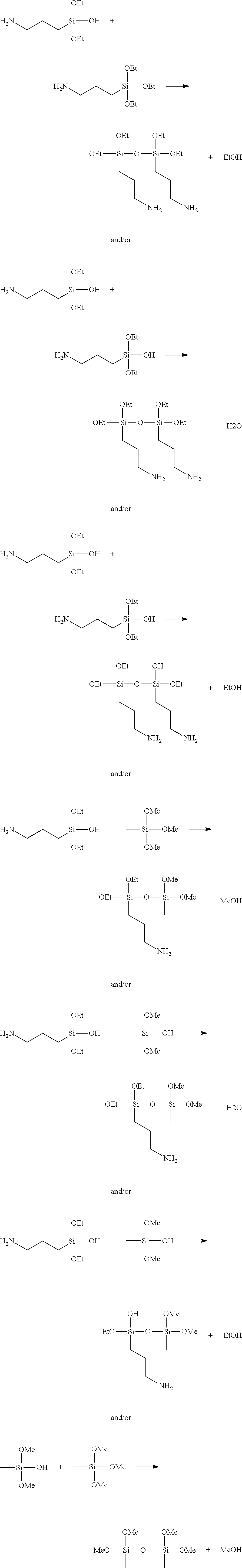

In the above exemplary reaction schemes the condensation to a dimer is shown in each case, but further condensations to oligomers with several silane atoms are also possible and preferred.

Both partially hydrolyzed and fully hydrolyzed C₁-C₆ alkoxysilanes of the formula (S-I) can participate in these condensation reactions, which undergo condensation with yet unreacted, partially or also fully hydrolyzed C₁-C₆ alkoxysilanes of the formula (S-I). In this case, the C₁-C₆ alkoxysilanes of formula (S-I) react with themselves.

Furthermore, both partially hydrolyzed and fully hydrolyzed C₁-C₆-alkoxysilanes of the formula (S-I) can also participate in the condensation reactions, which undergo condensation with not yet reacted, partially or also fully hydrolyzed C₁-C₆-alkoxysilanes of the formula (S-IV). In this case, the C₁-C₆ alkoxysilanes of formula (S-I) react with the C₁-C₆ alkoxysilanes of formula (S-IV).

Furthermore, both partially hydrolyzed and fully hydrolyzed C₁-C₆-alkoxysilanes of the formula (S-IV) can also participate in the condensation reactions, which undergo condensation with not yet reacted, partially or also fully hydrolyzed C₁-C₆-alkoxysilanes of the formula (S-IV). In this case, the C₁-C₆ alkoxysilanes of formula (S-IV) react with themselves.

Coloring with the Use of Pigments

In the process as contemplated herein, the decolorizing agent is applied to previously colored keratin material. In addition to the at least one organosilicon compound, the organic C₁-C₆ alkoxy-silane, at least one pigment is also used in the coloring process.

Pigments within the meaning of the present disclosure are coloring compounds which have a solubility in water at 25° C. of less than 0.5 g/L, preferably less than 0.1 g/L, even more preferably less than 0.05 g/L. Water solubility can be determined, for example, by the method described below: 0.5 g of the pigment are weighed in a beaker. A stir-fish is added. Then one liter of distilled water is added. This mixture is heated to 25° C. for one hour while stirring on a magnetic stirrer. If undissolved components of the pigment are still visible in the mixture after this period, the solubility of the pigment is below 0.5 g/L. If the pigment-water mixture cannot be assessed visually due to the high intensity of the finely dispersed pigment, the mixture is filtered. If a proportion of undissolved pigments remains on the filter paper, the solubility of the pigment is below 0.5 g/L.

Suitable color pigments can be of inorganic and/or organic origin.

In another very particularly preferred embodiment, the decolorizing agent is applied to keratin material which has been colored by application of at least one inorganic and/or organic pigment.

Preferred color pigments are selected from synthetic or natural inorganic pigments. Inorganic color pigments of natural origin can be produced, for example, from chalk, ochre, umber, green earth, burnt Terra di Siena or graphite. Furthermore, black pigments such as iron oxide black, colored pigments such as ultramarine or iron oxide red as well as fluorescent or phosphorescent pigments can be used as inorganic color pigments.

Particularly suitable are colored metal oxides, hydroxides and oxide hydrates, mixed-phase pigments, sulfur-comprising silicates, silicates, metal sulfides, complex metal cyanides, metal sulphates, chromates and/or molybdates. Preferred color pigments are black iron oxide (CI 77499), yellow iron oxide (CI 77492), red and brown iron oxide (CI 77491), manganese violet (CI 77742), ultramarine (sodium aluminum sulfo silicates, CI 77007, pigment blue 29), chromium oxide hydrate (CI77289), iron blue (ferric ferrocyanides, C177510) and/or carmine (cochineal).

As contemplated herein, colored pearlescent pigments are also particularly preferred color pigments. These are usually mica- and/or mica-based and can be coated with one or more metal oxides. Mica belongs to the layer silicates. The most important representatives of these silicates are muscovite, phlogopite, paragonite, biotite, lepidolite and margarite. To produce the pearlescent pigments in combination with metal oxides, the mica, muscovite or phlogopite, is coated with a metal oxide.

As an alternative to natural mica, synthetic mica coated with one or more metal oxides can also be used as pearlescent pigment. Especially preferred pearlescent pigments are based on natural or synthetic mica (mica) and are coated with one or more of the metal oxides mentioned above. The color of the respective pigments can be varied by varying the layer thickness of the metal oxide(s).

In another very particularly preferred embodiment, the decolorizing agent is applied to keratin material which has been colored by application of at least one pigment selected from the group of colored metal oxides, metal hydroxides, metal oxide hydrates, silicates, metal sulfides, complex metal cyanides, metal sulfates, bronze pigments and/or colored mica- or mica-based pigments coated with at least one metal oxide and/or a metal oxychloride.

In another very particularly preferred embodiment, the decolorizing agent is applied to keratin material which has been colored by application of at least one pigment selected from the group of mica- or mica-based pigments, colored with one or more metal oxides selected from the group of titanium dioxide (CI 77891), black iron oxide (CI 77499), yellow iron oxide (CI 77492), red and/or brown iron oxide (CI 77491, CI 77499), manganese violet (CI 77742), ultramarine (sodium aluminum sulfosilicates, CI 77007, Pigment Blue 29), chromium oxide hydrate (CI 77289), chromium oxide (CI 77288) and/or iron blue (ferric ferrocyanide, CI 77510).

Examples of particularly suitable color pigments are commercially available under the trade names Rona®, Colorona®, Xirona®, Dichrona® and Timiron® from Merck, Ariabel® and Unipure® from Sensient, Prestige® from Eckart Cosmetic Colors and Sunshine® from Sunstar.

Particularly preferred color pigments with the trade name Colorona® are, for example:

### Colorona Copper, Merck, MICA, CI 77491 (IRON OXIDES)

### Colorona Passion Orange, Merck, Mica, CI 77491 (Iron Oxides), Alumina

### Colorona Patina Silver, Merck, MICA, CI 77499 (IRON OXIDES), CI 77891 (TITANIUM DIOXIDE)

### Colorona RY, Merck, CI 77891 (TITANIUM DIOXIDE), MICA, CI 75470 (CARMINE)

### Colorona Oriental Beige, Merck, MICA, CI 77891 (TITANIUM DIOXIDE), CI 77491 (IRON OXIDES)

### Colorona Dark Blue, Merck, MICA, TITANIUM DIOXIDE, FERRIC FERROCYANIDE

### Colorona Chameleon, Merck, CI 77491 (IRON OXIDES), MICA

### Colorona Aborigine Amber, Merck, MICA, CI 77499 (IRON OXIDES), CI 77891 (TITANIUM DIOXIDE)

### Colorona Blackstar Blue, Merck, CI 77499 (IRON OXIDES), MICA

### Colorona Patagonian Purple, Merck, MICA, CI 77491 (IRON OXIDES), CI 77891 (TITANIUM DIOXIDE), CI 77510 (FERRIC FERROCYANIDE)

### Colorona Red Brown, Merck, MICA, CI 77491 (IRON OXIDES), CI 77891 (TITANIUM DIOXIDE)

### Colorona Russet, Merck, CI 77491 (TITANIUM DIOXIDE), MICA, CI 77891 (IRON OXIDES)

### Colorona Imperial Red, Merck, MICA, TITANIUM DIOXIDE (CI 77891), D&C RED NO. 30 (CI 73360)

### Colorona Majestic Green, Merck, CI 77891 (TITANIUM DIOXIDE), MICA, CI 77288 (CHROMIUM OXIDE GREENS)

### Colorona Light Blue, Merck, MICA, TITANIUM DIOXIDE (CI 77891), FERRIC FERROCYANIDE (CI 77510)

### Colorona Red Gold, Merck, MICA, CI 77891 (TITANIUM DIOXIDE), CI 77491 (IRON OXIDES)

### Colorona Gold Plus MP 25, Merck, MICA, TITANIUM DIOXIDE (CI 77891), IRON OXIDES (CI 77491)

### Colorona Carmine Red, Merck, MICA, TITANIUM DIOXIDE, CARMINE

### Colorona Blackstar Green, Merck, MICA, CI 77499 (IRON OXIDES)

### Colorona Bordeaux, Merck, MICA, CI 77491 (IRON OXIDES)

### Colorona Bronze, Merck, MICA, CI 77491 (IRON OXIDES)

### Colorona Bronze Fine, Merck, MICA, CI 77491 (IRON OXIDES)

### Colorona Fine Gold MP 20, Merck, MICA, CI 77891 (TITANIUM DIOXIDE), CI 77491 (IRON OXIDES)

### Colorona Sienna Fine, Merck, CI 77491 (IRON OXIDES), MICA

### Colorona Sienna, Merck, MICA, CI 77491 (IRON OXIDES)

Colorona Precious Gold, Merck, Mica, CI 77891 (Titanium dioxide), Silica, CI 77491 (Iron oxides), Tin oxide

### Colorona Sun Gold Sparkle MP 29, Merck, MICA, TITANIUM DIOXIDE, IRON OXIDES, MICA, CI 77891, CI 77491 (EU)

Colorona Mica Black, Merck, CI 77499 (Iron oxides), Mica, CI 77891 (Titanium dioxide)
Colorona Bright Gold, Merck, Mica, CI 77891 (Titanium dioxide), CI 77491 (Iron oxides)

### Colorona Blackstar Gold, Merck, MICA, CI 77499 (IRON OXIDES)

Other particularly preferred color pigments with the trade name Xirona® are for example:

### Xirona Golden Sky, Merck, Silica, CI 77891 (Titanium Dioxide), Tin Oxide

### Xirona Caribbean Blue, Merck, Mica, CI 77891 (Titanium Dioxide), Silica, Tin Oxide

### Xirona Kiwi Rose, Merck, Silica, CI 77891 (Titanium Dioxide), Tin Oxide

### Xirona Magic Mauve, Merck, Silica, CI 77891 (Titanium Dioxide), Tin Oxide.

In addition, particularly preferred color pigments with the trade name Unipure® are for example:

### Unipure Red LC 381 EM, Sensient CI 77491 (Iron Oxides), Silica

### Unipure Black LC 989 EM, Sensient, CI 77499 (Iron Oxides), Silica

### Unipure Yellow LC 182 EM, Sensient, CI 77492 (Iron Oxides), Silica

In a further embodiment of the process as contemplated herein, the keratin material may also have been dyed with at least one organic pigment prior to application of the decolorizing agent.

The organic pigments as contemplated herein are correspondingly insoluble, organic dyes or color lacquers, which may be selected, for example, from the group of nitroso, nitro-azo, xanthene, anthraquinone, isoindolinone, isoindolinone, quinacridone, perinone, perylene, diketo-pyrrolopyorrole, indigo, thioindido, dioxazine and/or triarylmethane compounds.

Examples of particularly suitable organic pigments are carmine, quinacridone, phthalocyanine, sorghum, blue pigments with the Color Index numbers C1 42090, CI 69800, CI 69825, CI 73000, CI 74100, CI 74160, yellow pigments with the Color Index numbers CI 11680, CI 11710, CI 15985, CI 19140, CI 20040, CI 21100, CI 21108, CI 47000, CI 47005, green pigments with the Color Index numbers CI 61565, CI 61570, CI 74260, orange pigments with the Color Index numbers CI 11725, CI 15510, CI 45370, CI 71105, red pigments with the Color Index numbers CI 12085, CI 12120, CI 12370, CI 12420, CI 12490, CI 14700, CI 15525, CI 15580, CI 15620, CI 15630, CI 15800, CI 15850, CI 15865, CI 15880, CI 17200, CI 26100, CI 45380, CI 45410, CI 58000, CI 73360, CI 73915 and/or CI 75470.

In another particularly preferred embodiment, the decolorizing agent is applied to keratin material which has been colored by application of at least one organic pigment selected from the group of carmine, quinacridone, phthalocyanine, sorghum, blue pigments having the color index numbers C1 42090, CI 69800, CI 69825, CI 73000, CI 74100, CI 74160, yellow pigments having the color index numbers CI 11680, CI 11710, CI 15985, CI 19140, CI 20040, CI 21100, CI 21108, CI 47000, CI 47005, green pigments with Color Index numbers CI 61565, CI 61570, CI 74260, orange pigments with Color Index numbers CI 11725, CI 15510, CI 45370, CI 71105, red pigments with the Color Index numbers CI 12085, CI 12120, CI 12370, CI 12420, CI 12490, CI 14700, CI 15525, CI 15580, CI 15620, CI 15630, CI 15800, CI 15850, CI 15865, CI 15880, CI 17200, CI 26100, CI 45380, CI 45410, CI 58000, CI 73360, CI 73915 and/or CI 75470.

The organic pigment can also be a color paint. In the context of the present disclosure, the term color varnish is understood to mean particles comprising a layer of absorbed dyes, the unit of particle and dye being insoluble under the above conditions. The particles can, for example, be inorganic substrates, which can be aluminum, silica, calcium borosilate, calcium aluminum borosilicate or even aluminum.

For example, alizarin color varnish can be used.

Due to their excellent light and temperature resistance, coloring with the pigments is particularly preferred. It is also preferred if the pigments used have a certain particle size. This particle size leads on the one hand to an even distribution of the pigments in the formed polymer film and on the other hand avoids a rough hair or skin feeling after application of the cosmetic product. As contemplated herein, it is therefore advantageous if the at least one pigment has an average particle size D₅₀ of 1.0 to 50 μm, preferably 5.0 to 45 μm, preferably 10 to 40 μm, 14 to 30 μm. The mean particle size D₅₀, for example, can be determined using dynamic light scattering (DLS).

Pigments with a specific shaping may also have been used to color the keratin material. For example, a pigment based on a lamellar and/or a lenticular substrate platelet can be used. Furthermore, coloring based on a substrate platelet comprising a vacuum metallized pigment is also possible.

The substrate platelets of this type have an average thickness of at most 50 nm, preferably less than 30 nm, particularly preferably at most 25 nm, for example at most 20 nm. The average thickness of the substrate platelets is at least 1 nm, preferably at least 2.5 nm, particularly preferably at least 5 nm, for example at least 10 nm. Preferred ranges for substrate wafer thickness are 2.5 to 50 nm, 5 to 50 nm, 10 to 50 nm; 2.5 to 30 nm, 5 to 30 nm, 10 to 30 nm; 2.5 to 25 nm, 5 to 25 nm, 10 to 25 nm, 2.5 to 20 nm, 5 to 20 nm, and 10 to 20 nm. Preferably, each substrate plate has a thickness that is as uniform as possible.

Due to the low thickness of the substrate platelets, the pigment exhibits particularly high hiding power.

The substrate plates have a monolithic structure. Monolithic in this context means comprising a single closed unit without fractures, stratifications or inclusions, although structural changes may occur within the substrate platelets. The substrate platelets are preferably homogeneously structured, i.e., there is no concentration gradient within the platelets. In particular, the substrate platelets do not have a layered structure and do not have any particles or particles distributed in them.

The size of the substrate platelet can be adjusted to the respective application purpose, especially the desired effect on the keratinic material. Typically, the substrate platelets have an average largest diameter of about 2 to 200 μm, especially about 5 to 100 μm.

In a preferred design, the aspect ratio, expressed by the ratio of the average size to the average thickness, is at least 80, preferably at least 200, more preferably at least 500, more preferably more than 750. The average size of the uncoated substrate platelets is the d50 value of the uncoated substrate platelets. Unless otherwise stated, the d50 value was determined using a Sympatec Helos device with quixel wet dispersion. To prepare the sample, the sample to be analyzed was pre-dispersed in isopropanol for 3 minutes.

The substrate platelets can be composed of any material that can be formed into platelet shape.

They can be of natural origin, but also synthetically produced. Materials from which the substrate platelets can be constructed include metals and metal alloys, metal oxides, preferably aluminum oxide, inorganic compounds and minerals such as mica and (semi-)precious stones, and plastics. Preferably, the substrate platelets are constructed of metal (alloy).

Any metal suitable for metallic luster pigments can be used. Such metals include iron and steel, as well as all air and water resistant (semi)metals such as platinum, zinc, chromium, molybdenum and silicon, and their alloys such as aluminum bronzes and brass. Preferred metals are aluminum, copper, silver and gold. Preferred substrate platelets include aluminum platelets and brass platelets, with aluminum substrate platelets being particularly preferred.

Lamellar substrate platelets are exemplified by an irregularly structured edge and are also referred to as “cornflakes” due to their appearance.

Due to their irregular structure, pigments based on lamellar substrate platelets generate a high proportion of scattered light. In addition, pigments based on lamellar substrate platelets do not completely cover the existing color of a keratinous material, and effects analogous to natural graying can be achieved, for example.

Lenticular (=lens-shaped) substrate platelets have a regular round edge and are also called “silver dollars” due to their appearance. Due to their regular structure, the proportion of reflected light predominates in pigments based on lenticular substrate platelets.

Vacuum metallized pigments (VMP) can be obtained, for example, by releasing metals, metal alloys or metal oxides from suitably coated films. They are exemplified by a particularly low thickness of the substrate platelets in the range of 5 to 50 nm and a particularly smooth surface with increased reflectivity. Substrate platelets comprising a vacuum metallized pigment are also referred to as VMP substrate platelets in the context of this application. VMP substrate platelets of aluminum can be obtained, for example, by releasing aluminum from metallized films.

The metal or metal alloy substrate plates can be passivated, for example by anodizing (oxide layer) or chromating.

Uncoated lamellar, lenticular and/or VPM substrate plates, especially those made of metal or metal alloy, reflect the incident light to a high degree and create a light-dark flop but no color impression.

A color impression can be created by optical interference effects, for example. Such pigments can be based on at least single-coated substrate platelets. These show interference effects by superimposing differently refracted and reflected light beams.

Accordingly, preferred pigments, pigments based on a coated lamellar substrate platelet. The substrate wafer preferably has at least one coating B of a highly refractive metal oxide having a coating thickness of at least 50 nm. There is preferably another coating A between the coating B and the surface of the substrate wafer. If necessary, there is a further coating C on the layer B, which is different from the layer B underneath.

Suitable materials for coatings A, B and C are all substances that can be applied to the substrate platelets in a film-like and permanent manner and, in the case of coatings A and B, have the required optical properties. Coating part of the surface of the substrate platelets is sufficient to obtain a pigment with a glossy effect. For example, only the top and/or bottom of the substrate platelets may be coated, with the side surface(s) omitted. Preferably, the entire surface of the optionally passivated substrate platelets, including the side surfaces, is covered by coating B. The substrate platelets are thus completely enveloped by coating B. This improves the optical properties of the pigment and increases its mechanical and chemical resistance. The above also applies to layer A and preferably also to layer C, if present.

Although multiple coatings A, B and/or C may be present in each case, the coated substrate wafers preferably have only one coating A, B and, if present, C in each case.

The coating B is composed of at least one highly refractive metal oxide. Highly refractive materials have a refractive index of at least 1.9, preferably at least 2.0, and more preferably at least 2.4. Preferably, the coating B comprises at least 95 wt. %, more preferably at least 99 wt. %, of high refractive index metal oxide(s).

The coating B has a thickness of at least 50 nm. Preferably, the thickness of coating B is no more than 400 nm, more preferably no more than 300 nm.

Highly refractive metal oxides suitable for coating B are preferably selectively light-absorbing (i.e., colored) metal oxides, such as iron(III) oxide (α- and γ-Fe₂O₃, red), cobalt(II) oxide (blue), chromium(III) oxide (green), titanium(III) oxide (blue, usually present in admixture with titanium oxynitrides and titanium nitrides), and vanadium(V) oxide (orange), and mixtures thereof. Colorless high-index oxides such as titanium dioxide and/or zirconium oxide are also suitable.

Coating B may comprise a selectively absorbing dye, preferably 0.001 to 5% by weight, particularly preferably 0.01 to 1% by weight, in each case based on the total amount of coating B. Suitable dyes are organic and inorganic dyes which can be stably incorporated into a metal oxide coating.

The coating A preferably has at least one low refractive index metal oxide and/or metal oxide hydrate. Preferably, coating A comprises at least 95 wt. %, more preferably at least 99 wt. %, of low refractive index metal oxide (hydrate). Low refractive index materials have a refractive index of 1.8 or less, preferably 1.6 or less.

Low refractive index metal oxides suitable for coating A include, for example, silicon (di)oxide, silicon oxide hydrate, aluminum oxide, aluminum oxide hydrate, boron oxide, germanium oxide, manganese oxide, magnesium oxide, and mixtures thereof, with silicon dioxide being preferred. The coating A preferably has a thickness of 1 to 100 nm, particularly preferably 5 to 50 nm, especially preferably 5 to 20 nm.

Preferably, the distance between the surface of the substrate platelets and the inner surface of coating B is at most 100 nm, particularly preferably at most 50 nm, especially preferably at most 20 nm. By ensuring that the thickness of coating A, and thus the distance between the surface of the substrate platelets and coating B, is within the range specified above, it is possible to ensure that the pigments have a high hiding power.

If the pigment based on a lamellar substrate platelet has only one layer A, it is preferred that the pigment has a lamellar substrate platelet of aluminum and a layer A of silica. If the pigment based on a lamellar substrate platelet has a layer A and a layer B, it is preferred that the pigment has a lamellar substrate platelet of aluminum, a layer A of silica and a layer B of iron oxide.

According to a preferred embodiment, the pigments have a further coating C of a metal oxide (hydrate), which is different from the underlying coating B. Suitable metal oxides include silicon (di)oxide, silicon oxide hydrate, aluminum oxide, aluminum oxide hydrate, zinc oxide, tin oxide, titanium dioxide, zirconium oxide, iron (III) oxide, and chromium (III) oxide. Silicon dioxide is preferred.

The coating C preferably has a thickness of 10 to 500 nm, more preferably 50 to 300 nm. By providing coating C, for example based on TiO₂, better interference can be achieved while maintaining high hiding power.

Layers A and C serve as corrosion protection as well as chemical and physical stabilization. Particularly preferred layers A and C are silica or alumina applied by the sol-gel process. This process comprises dispersing the uncoated lamellar substrate platelets or the lamellar substrate platelets already coated with layer A and/or layer B in a solution of a metal alkoxide such as tetraethyl orthosilicate or aluminum triisopropanolate (usually in a solution of organic solvent or a mixture of organic solvent and water with at least 50% by weight of organic solvent such as a C1 to C4 alcohol) and adding a weak base or acid to hydrolyze the metal alkoxide. % Organic solvent such as a C1 to C4 alcohol) and adding a weak base or acid to hydrolyze the metal alkoxide, thereby forming a film of the metal oxide on the surface of the (coated) substrate platelets.

Layer B can be produced, for example, by hydrolytic decomposition of one or more organic metal compounds and/or by precipitation of one or more dissolved metal salts, as well as any subsequent post-treatment (for example, transfer of a formed hydroxide-containing layer to the oxide layers by annealing).

Although each of the coatings A, B and/or C may be composed of a mixture of two or more metal oxide(hydrate)s, each of the coatings is preferably composed of one metal oxide(hydrate).

The pigments based on coated lamellar or lenticular substrate platelets, or the pigments based on coated VMP substrate platelets preferably have a thickness of 70 to 500 nm, particularly preferably 100 to 400 nm, especially preferably 150 to 320 nm, for example 180 to 290 nm. Due to the low thickness of the substrate platelets, the pigment exhibits particularly high hiding power. The low thickness of the coated substrate platelets is achieved by keeping the thickness of the uncoated substrate platelets low, but also by adjusting the thicknesses of the coatings A and, if present, C to as small a value as possible. The thickness of coating B determines the color impression of the pigment.

The adhesion and abrasion resistance of pigments based on coated substrate platelets in keratinic material can be significantly increased by additionally modifying the outermost layer, layer A, B or C depending on the structure, with organic compounds such as silanes, phosphoric acid esters, titanates, borates or carboxylic acids. In this case, the organic compounds are bonded to the surface of the outermost, preferably metal oxide-containing, layer A, B, or C. The outermost layer denotes the layer that is spatially farthest from the lamellar substrate platelet. The organic compounds are preferably functional silane compounds that can bind to the metal oxide-containing layer A, B, or C. These can be either mono- or bifunctional compounds. Examples of bifunctional organic compounds include methacryloxypropenyltrimethoxysilane, 3-methacryloxypropyltrimethoxysilane, 3-acryloxypropyltrimethoxysilane, 2-acryloxyethyltrimethoxysilane, 3-methacryloxy-propyltriethoxysilane, 3-acryloxypropyltrimethoxysilane, 2-methacryloxyethyl-triethoxysilane, 2-acryloxyethyltriethoxysilane, 3-methacryloxypropyltris(methoxyethoxy)silane, 3-methacryloxypropyltris(butoxyethoxy)silane, 3-methacryloxy-propyltris(propoxy)silane, 3-methacryloxypropyltris(butoxy)silane, 3-acryloxy-propyltris(methoxyethoxy)silane, 3-acryloxypropyltris(butoxyethoxy)silane, 3-acryl-oxypropyltris(butoxy)silane, vinyltrimethoxysilane, vinyltriethoxysilane, vinylethyl dichlorosilane, vinylmethyldiacetoxysilane, vinylmethyldichlorosilane, vinylmethyldiethoxysilane, vinyltriacetoxysilane, vinyltrichlorosilane, phenylvinyldiethoxysilane, or phenylallyldichlorosilane. Furthermore, a modification with a monofunctional silane, an alkyl silane or arylsilane, can be carried out. This has only one functional group, which can covalently bond to the surface pigment based on coated lamellar substrate platelets (i.e., to the outermost metal oxide-containing layer) or, if not completely covered, to the metal surface. The hydrocarbon residue of the silane points away from the pigment. Depending on the type and nature of the hydrocarbon residue of the silane, a varying degree of hydrophobicity of the pigment is achieved. Examples of such silanes include hexadecyltrimethoxysilane, propyltrimethoxysilane, etc. Particularly preferred are pigments based on silica-coated aluminum substrate platelets surface-modified with a monofunctional silane. Octyltrimethoxysilane, octyltriethoxysilane, hexadecyltrimethoxysilane and hexadecyltriethoxysilane are particularly preferred. Due to the changed surface properties/hydrophobization, an improvement can be achieved in terms of adhesion, abrasion resistance and alignment in the application.

Suitable pigments based on a lamellar substrate platelet include, for example, the pigments of the VISIONAIRE series from Eckart.

Pigments based on a lenticular substrate platelet are available, for example, under the name Alegrace® Gorgeous from the company Schlenk Metallic Pigments GmbH.

Pigments based on a substrate platelet comprising a vacuum metallized pigment are available, for example, under the name Alegrace® Marvelous or Alegrace® Aurous from the company Schlenk Metallic Pigments GmbH.

Dyeing with the Use of Polymers

In addition to the organosilicon compound(s), in particular the organic C₁-C₆ alkoxy silanes and the pigments, at least one film-forming polymer can also be used in the coloring of the keratin material, in particular the keratin fibers.

It has been found that the colorations obtained by applying the combination of organic C₁-C₆ alkoxy-silane, pigment and film-forming polymer are particularly resistant and therefore especially difficult to decolorize. Surprisingly, it has been found that the application of the decolorizing agent as contemplated herein is also able to decolorize these dyeings excellently.

In another very particularly preferred embodiment, the decolorizing agent is applied to keratin material which has been colored by application of at least one organic C₁-C₆ alkoxy-silane and/or a condensation product thereof, by application of at least one pigment and by application of at least one film-forming polymer.

Polymers are macromolecules with a molecular weight of at least 1000 g/mol, preferably of at least 2500 g/mol, particularly preferably of at least 5000 g/mol, which include identical, repeating organic units. The polymers of the present disclosure may be synthetically produced polymers which are manufactured by polymerization of one type of monomer or by polymerization of several types of monomer which are structurally different from each other. If the polymer is produced by polymerizing a type of monomer, it is called a homo-polymer. If structurally different monomer types are used in polymerization, the resulting polymer is called a copolymer.

The maximum molecular weight of the polymer depends on the degree of polymerization (number of polymerized monomers) and the batch size and is determined by the polymerization method. For the purposes of the present disclosure, it is preferred that the maximum molecular weight of the film-forming hydrophobic polymer (c) is not more than 107 g/mol, preferably not more than 10⁶ g/mol and particularly preferably not more than 105 g/mol.

As contemplated herein, a film-forming polymer is a polymer which can form a film on a substrate, for example on a keratinic material or a keratinic fiber. The formation of a film can be demonstrated, for example, by looking at the keratin material treated with the polymer under a microscope.

The film-forming polymers previously applied in the dyeing step can be hydrophilic or hydrophobic.

In another very particularly preferred embodiment, the decolorizing agent is applied to keratin material which has been colored by application of at least one organic C₁-C₆ alkoxy-silane and/or a condensation product thereof, by application of at least one pigment and by application of at least one film-forming, hydrophobic polymer.

A hydrophobic polymer is a polymer that has a solubility in water at 25° C. (760 mmHg) of less than 1% by weight.

The water solubility of the film-forming, hydrophobic polymer can be determined in the following way, for example. 1.0 g of the polymer is placed in a beaker. Make up to 100 g with water. A stir-fish is added, and the mixture is heated to 25° C. on a magnetic stirrer while stirring. It is stirred for 60 minutes. The aqueous mixture is then visually assessed. If the polymer-water mixture cannot be assessed visually due to a high turbidity of the mixture, the mixture is filtered. If a proportion of undissolved polymer remains on the filter paper, the solubility of the polymer is less than 1% by weight.

These include acrylic acid-type polymers, polyurethanes, polyesters, polyamides, polyureas, cellulose polymers, nitrocellulose polymers, silicone polymers, acrylamide-type polymers and polyisoprenes.

Particularly well suited film-forming, hydrophobic polymers are, for example, polymers from the group of copolymers of acrylic acid, copolymers of methacrylic acid, homopolymers or copolymers of acrylic acid esters, homopolymers or copolymers of methacrylic acid esters, homopolymers or copolymers of acrylic acid amides, homopolymers or copolymers of methacrylic acid amides, copolymers of vinylpyrrolidone, copolymers of vinyl alcohol, copolymers of vinyl acetate, homopolymers or copolymers of ethylene, homopolymers or copolymers of propylene, homopolymers or copolymers of styrene, polyurethanes, polyesters and/or polyamides.

In a further particularly preferred embodiment, the decolorizing agent is applied to keratin material which has been colored by application of at least one film-forming, hydrophobic polymer selected from the group of copolymers of acrylic acid, copolymers of methacrylic acid, homopolymers or copolymers of acrylic acid esters, homopolymers or copolymers of methacrylic acid esters, homopolymers or copolymers of acrylic acid amides, homopolymers or copolymers of methacrylic acid amides, copolymers of vinylpyrrolidone, copolymers of vinyl alcohol, copolymers of vinyl acetate, homopolymers or copolymers of ethylene, homopolymers or copolymers of propylene, homopolymers or copolymers of styrene, polyurethanes, polyesters and/or polyamides.

The film-forming hydrophobic polymers, which are selected from the group of synthetic polymers, polymers obtainable by radical polymerization or natural polymers, have proved to be particularly suitable for solving the problem as contemplated herein.

Other particularly well-suited film-forming hydrophobic polymers can be selected from the homopolymers or copolymers of olefins, such as cycloolefins, butadiene, isoprene or styrene, vinyl ethers, vinyl amides, the esters or amides of (meth)acrylic acid having at least one C₁-C₂₀ alkyl group, an aryl group or a C₂-C₁₀ hydroxyalkyl group.

Other film-forming hydrophobic polymers may be selected from the homo- or copolymers of isooctyl (meth)acrylate; isonononyl (meth)acrylate; 2-ethylhexyl (meth)acrylate; lauryl (meth)acrylate; isopentyl (meth)acrylate; n-butyl (meth)acrylate); isobutyl (meth)acrylate; ethyl (meth)acrylate; methyl (meth)acrylate; tert-butyl (meth)acrylate; stearyl (meth)acrylate; hydroxyethyl (meth)acrylate; 2-hydroxypropyl (meth)acrylate; 3-hydroxypropyl (meth)acrylate and/or mixtures thereof.

Other film-forming hydrophobic polymers may be selected from the homo- or copolymers of (meth)acrylamide; N-alkyl-(meth)acrylamides, in those with C2-C18 alkyl groups, such as N-ethyl-acrylamide, N-tert-butyl-acrylamide, N-octyl-acrylamide; N-di(C1-C4)alkyl-(meth)acrylamide.

Other preferred anionic copolymers are, for example, copolymers of acrylic acid, methacrylic acid or their C₁-C₆ alkyl esters, as they are marketed under the INCI Declaration Acrylates Copolymers. A suitable commercial product is for example Aculyn® 33 from Rohm & Haas. Copolymers of acrylic acid, methacrylic acid or their C₁-C₆ alkyl esters and the esters of an ethylenically unsaturated acid and an alkoxylated fatty alcohol are also preferred. Suitable ethylenically unsaturated acids are especially acrylic acid, methacrylic acid and itaconic acid; suitable alkoxylated fatty alcohols are especially steareth-20 or ceteth-20.

Very particularly preferred polymers on the market are, for example, Aculyn® 22 (Acrylates/Steareth-20 Methacrylate Copolymer), Aculyn® 28 (Acrylates/Beheneth-25 Methacrylate Copolymer), Structure 2001@ (Acrylates/Steareth-20 Itaconate Copolymer), Structure 3001@ (Acrylates/Ceteth-20 Itaconate Copolymer), Structure Plus® (Acrylates/Aminoacrylates C10-30 Alkyl PEG-20 Itaconate Copolymer), Carbopol® 1342, 1382, Ultrez 20, Ultrez 21 (Acrylates/C10-30 Alkyl Acrylate Crosspolymer), Synthalen W 2000® (Acrylates/Palmeth-25 Acrylate Copolymer) or the Rohme und Haas distributed Soltex OPT (Acrylates/C₁₂₋₂₂ Alkyl methacrylate Copolymer).

The homo- and copolymers of N-vinylpyrrolidone, vinylcaprolactam, vinyl-(C1-C6)alkyl-pyrrole, vinyl-oxazole, vinyl-thiazole, vinylpyrimidine, vinylimidazole can be named as suitable polymers based on vinyl monomers.

Furthermore, the copolymers octylacrylamide/acrylates/butylaminoethyl-methacrylate copolymer, as commercially marketed under the trade names AMPHOMER® or LOVOCRYL® 47 by NATIONAL STARCH, or the copolymers of acrylates/octylacrylamides marketed under the trade names DERMACRYL® LT and DERMACRYL® 79 by NATIONAL STARCH are particularly suitable.

Suitable olefin-based polymers include homopolymers and copolymers of ethylene, propylene, butene, isoprene and butadiene.

In another embodiment, the film-forming hydrophobic polymers may be the block copolymers comprising at least one block of styrene or the derivatives of styrene. These block copolymers can be copolymers that comprise one or more other blocks in addition to a styrene block, such as styrene/ethylene, styrene/ethylene/butylene, styrene/butylene, styrene/isoprene, styrene/butadiene. Such polymers are commercially distributed by BASF under the trade name “Luvitol HSB”.

In another embodiment, the film-forming polymers previously applied in the dyeing step may also be hydrophilic.

In another very particularly preferred embodiment, the decolorizing agent is applied to keratin material which has been colored by application of at least one organic C₁-C₆ alkoxy-silane and/or a condensation product thereof, by application of at least one pigment and by application of at least one film-forming, hydrophilic polymer.

A hydrophilic polymer is a polymer that has a solubility in water at 25° C. (760 mmHg) of more than 1% by weight, preferably more than 2% by weight.

The water solubility of the film-forming, hydrophilic polymer can be determined in the following way, for example. 1.0 g of the polymer is placed in a beaker. Make up to 100 g with water. A stir-fish is added, and the mixture is heated to 25° C. on a magnetic stirrer while stirring. It is stirred for 60 minutes. The aqueous mixture is then visually assessed. A completely dissolved polymer appears macroscopically homogeneous. If the polymer-water mixture cannot be assessed visually due to a high turbidity of the mixture, the mixture is filtered. If no undissolved polymer remains on the filter paper, the solubility of the polymer is more than 1% by weight.

Nonionic, anionic and cationic polymers can be used as film-forming, hydrophilic polymers.

Suitable film-forming hydrophilic polymers can be selected, for example, from the group of polyvinylpyrrolidone (co)polymers, polyvinyl alcohol (co)polymers, vinyl acetate (co)polymers, carboxyvinyl (co)polymers, acrylic acid (co)polymers, methacrylic acid (co)polymers, natural gums, polysaccharides and/or acrylamide (co)polymers.

Furthermore, it is particularly preferred to use polyvinylpyrrolidone (PVP) and/or a vinylpyrrolidone-containing copolymer as film-forming hydrophilic polymer.

In another very particularly preferred embodiment, the decolorizing agent is applied to keratin material which has been colored by application of at least one film-forming hydrophilic polymer selected from the group of polyvinylpyrrolidone (PVP) and the copolymers of polyvinylpyrrolidone.

It is further preferred if the colorant comprises polyvinylpyrrolidone (PVP) as the film-forming hydrophilic polymer. Particularly well-suited polyvinylpyrrolidones are available, for example, under the name Luviskol® K from BASF SE, especially Luviskol® K 90 or Luviskol® K 85 from BASF SE.

The polymer PVP K30, which is marketed by Ashland (ISP, POI Chemical), can also be used as another explicitly very well suited polyvinylpyrrolidone (PVP). PVP K 30 is a polyvinylpyrrolidone which is highly soluble in cold water and has the CAS number 9003-39-8. The molecular weight of PVP K 30 is about 40000 g/mol.

Other particularly suitable polyvinylpyrrolidones are the substances known under the trade names LUVITEC K 17, LUVITEC K 30, LUVITEC K 60, LUVITEC K 80, LUVITEC K 85, LUVITEC K 90 and LUVITEC K 115 and available from BASF.

The use of film-forming hydrophilic polymers from the group of copolymers of polyvinylpyrrolidone has also led to particularly satisfactory results.

Vinylpyrrolidone-vinyl ester copolymers, such as those marketed under the trademark Luviskol® (BASF), are particularly suitable film-forming hydrophilic polymers. Luviskol® VA 64 and Luviskol® VA 73, both vinylpyrrolidone/vinyl acetate copolymers, are particularly preferred non-ionic polymers.

Of the vinylpyrrolidone-containing copolymers, a styrene/VP copolymer and/or a vinylpyrrolidone-vinyl acetate copolymer and/or a VP/DMAPA acrylates copolymer and/or a VP/vinyl caprolactam/DMAPA acrylates copolymer are particularly preferred in cosmetic compositions.

Vinylpyrrolidone-vinyl acetate copolymers are marketed under the name Luviskol® VA by BASF SE. For example, a VP/Vinyl Caprolactam/DMAPA Acrylates copolymer is sold under the trade name Aquaflex® SF-40 by Ashland Inc. For example, a VP/DMAPA acrylates copolymer is marketed by Ashland under the name Styleze CC-10 and is a highly preferred vinylpyrrolidone-containing copolymer.

Other suitable copolymers of polyvinylpyrrolidone may also be those obtained by reacting N-vinylpyrrolidone with at least one further monomer from the group comprising V-vinylformamide, vinyl acetate, ethylene, propylene, acrylamide, vinylcaprolactam, vinylcaprolactone and/or vinyl alcohol.

In another very particularly preferred embodiment, the decolorizing agent is applied to keratin material which has been colored by application of at least one film-forming hydrophilic polymer selected from the group of polyvinylpyrrolidone (PVP), vinylpyrrolidone/vinyl acetate copolymers, vinylpyrrolidone/styrene copolymers, vinylpyrrolidone/ethylene copolymers, vinylpyrrolidone/propylene copolymers, vinylpyrrolidone/vinylcaprolactam copolymers, vinylpyrrolidone/vinylformamide copolymers and/or vinylpyrrolidone/vinyl alcohol copolymers.

Another suitable copolymer of vinylpyrrolidone is the polymer known under the INCI designation maltodextrin/VP copolymer.

Furthermore, intensively colored keratin material, especially hair, could be recolored with particularly satisfactory results when a nonionic, film-forming, hydrophilic polymer was used as the film-forming, hydrophilic polymer.

In a first embodiment, it may be preferred if the keratin material has been dyed with at least one non-ionic, film-forming, hydrophilic polymer in the previous dyeing step.

As contemplated herein, a non-ionic polymer is understood to be a polymer which in a protic solvent—such as water—under standard conditions does not carry structural units with permanent cationic or anionic groups, which must be compensated by counterions while maintaining electron neutrality. Cationic groups include quaternized ammonium groups but not protonated amines. Anionic groups include carboxylic and sulphonic acid groups.

The colorants which comprise as nonionic, film-forming, hydrophilic polymer at least one polymer selected from the group of
Polyvinylpyrrolidone,
Copolymers of N-vinylpyrrolidone and vinyl esters of carboxylic acids having 2 to 18 carbon atoms of N-vinylpyrrolidone and vinyl acetate,
Copolymers of N-vinylpyrrolidone and N-vinylimidazole and methacrylamide,
Copolymers of N-vinylpyrrolidone and N-vinylimidazole and acrylamide,
Copolymers of N-vinylpyrrolidone with N,N-di(C1 to C4)-alkylamino-(C2 to C4)-alkylacrylamide,

If copolymers of N-vinylpyrrolidone and vinyl acetate are used, it is again preferable if the molar ratio of the structural units included in the monomer N-vinylpyrrolidone to the structural units of the polymer included in the monomer vinyl acetate is in the range from 20:80 to 80:20, in particular from 30:70 to 60:40. Suitable copolymers of vinyl pyrrolidone and vinyl acetate are available, for example, under the trademarks Luviskol® VA 37, Luviskol® VA 55, Luviskol® VA 64 and Luviskol® VA 73 from BASF SE.

Another particularly preferred polymer is selected from the INCI designation VP/Methacrylamide/Vinyl Imidazole Copolymer, which is available under the trade name Luviset Clear from BASF SE.

Another very particularly preferred nonionic, film-forming, hydrophilic polymer is a copolymer of N-vinylpyrrolidone and N,N-dimethylaminiopropylmethacrylamide, which is sold, for example, by the company ISP under the INCI designation VP/DMAPA Acrylates Copolymer, e.g., under the trade name Styleze® CC 10.

A cationic polymer of interest is the copolymer of N-vinylpyrrolidone, N-vinylcaprolactam, N-(3-dimethylaminopropyl)methacrylamide and 3-(methacryloylamino)propyl-lauryl-dimethylammonium chloride (INCI designation): Polyquaternium-69), which is marketed, for example, under the trade name AquaStyle® 300 (28-32 wt. % active substance in ethanol-water mixture, molecular weight 350000) by ISP.

Other suitable film-forming, hydrophilic polymers include
Vinylpyrrolidone-vinylimidazolium methochloride copolymers, as offered under the designations Luviquat® FC 370, FC 550 and the INCI designation Polyquaternium-16 as well as FC 905 and HM 552,
Vinylpyrrolidone-vinylcaprolactam-acrylate terpolymers, as they are commercially available with acrylic acid esters and acrylic acid amides as a third monomer component, for example under the name Aquaflex® SF 40.

Polyquaternium-11 is the reaction product of diethyl sulphate with a copolymer of vinyl pyrrolidone and dimethylaminoethyl methacrylate. Suitable commercial products are available under the names Dehyquart® CC 11 and Luviquat® PQ 11 PN from BASF SE or Gafquat 440, Gafquat 734, Gafquat 755 or Gafquat 755N from Ashland Inc.

Polyquaternium-46 is the reaction product of vinylcaprolactam and vinylpyrrolidone with methylvinylimidazolium methosulfate and is available for example under the name Luviquat® Hold from BASF SE. Polyquaternium-46 is preferably used in an amount of 1 to 5% by weight—based on the total weight of the cosmetic composition. It particularly prefers to use polyquaternium-46 in combination with a cationic guar compound. It is even highly preferred that polyquaternium-46 is used in combination with a cationic guar compound and polyquaternium-11.

Suitable anionic film-forming, hydrophilic polymers can be, for example, acrylic acid polymers, which can be in non-crosslinked or crosslinked form. Such products are sold commercially under the trade names Carbopol 980, 981, 954, 2984 and 5984 by Lubrizol or under the names Synthalen M and Synthalen K by 3V Sigma (The Sun Chemicals, Inter Harz).

Examples of suitable film-forming, hydrophilic polymers from the group of natural gums are xanthan gum, gellan gum, carob gum.

Examples of suitable film-forming hydrophilic polymers from the group of polysaccharides are hydroxyethyl cellulose, hydroxypropyl cellulose, ethyl cellulose and carboxymethyl cellulose.

Suitable film-forming, hydrophilic polymers from the acrylamide group are, for example, polymers prepared from monomers of (Methyl)acrylamido-C1-C4-alkyl-sulfonic acid or salts thereof. Corresponding polymers may be selected from the polymers of polyacrylamidomethanesulfonic acid, polyacrylamidoethanesulfonic acid, polyacrylamidopropanesulfonic acid, poly2-acrylamido-2-methylpropanesulfonic acid, poly-2-methylacrylamido-2-methylpropanesulfonic acid and/or poly-2-methylacrylamido-n-butanesulfonic acid.

Preferred polymers of poly(meth)arylamido-C₁-C₄-alkyl-sulfonic acids are crosslinked and at least 90% neutralized. These polymers can be crosslinked or non-crosslinked.

Cross-linked and fully or partially neutralized polymers of the poly-2-acrylamido-2-methylpropane sulfonic acid type are available under the INCI names “Ammonium Polyacrylamido-2-methyl-propanesulphonates” or “Ammonium Poly acryldimethyltauramides”.

Another preferred polymer of this type is the cross-linked poly-2-acrylamido-2-methyl-propanesulphonic acid polymer marketed by Clamant under the trade name Hostacerin AMPS, which is partially neutralized with ammonia.

In another very particularly preferred embodiment, the decolorizing agent is applied to keratin material which has been colored by application of at least one organic C₁-C₆ alkoxy-silane and/or a condensation product thereof, by application of at least one pigment and by application of at least one film-forming, anionic polymer.

In this context, the best results have been obtained when the decolorizing agent is applied to keratin material which has been colored by application of at least one organic C₁-C₆ alkoxy-silane and/or a condensation product thereof, by application of at least one pigment and by application of at least one film-forming anionic polymer, said film-forming anionic polymer comprising at least one structural unit of formula (P-I) and at least one structural unit of formula (P-II)

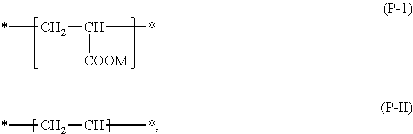

where
M is a hydrogen atom or ammonium (NH₄), sodium, potassium, ½ magnesium or 12 calcium.

When M represents a hydrogen atom, the structural unit of the formula (P-I) is based on an acrylic acid unit.

When M stands for an ammonium counterion, the structural unit of the formula (P-I) is based on the ammonium salt of acrylic acid.
When M stands for a sodium counterion, the structural unit of the formula (P-I) is based on the sodium salt of acrylic acid.
When M stands for a potassium counterion, the structural unit of the formula (P-I) is based on the potassium salt of acrylic acid.
If M stands for a half equivalent of a magnesium counterion, the structural unit of the formula (P-I) is based on the magnesium salt of acrylic acid.
If M stands for a half equivalent of a calcium counterion, the structural unit of the formula (P-I) is based on the calcium salt of acrylic acid.

### Application of the Decolorizing Agent

In the process as contemplated herein, the decolorizing agent is applied to the colored keratin material and rinsed off again after a contact time.

Since the decolorizing agent is applied to the colored hair, the decolorizing agent must be applied to the keratin material after the application of the previously described colorant.

In other words, the decolorizing agent is applied to the keratin material after the colorant has been rinsed out and the keratin material has preferably been dried to accurately determine the color result.

The exact time of application of the decolorizing agent is determined by the user's wish to remove the unwanted or no longer required coloration. For example, the decolorizing agent can be applied to the dyed keratin material 12 to 24 hours after application of the dyeing agent. In a further embodiment, however, the user may wear the colored keratin materials, the hair, for a period of several days to weeks until the user decides to change the coloration again or the user wants to have his original hair color back.

The decolorizing agent is exemplified by its content of at least one solvent (a) and at least one alkalizing agent (b).

### Solvent (a)

As a first ingredient essential to the present disclosure, the decolorizing agent comprises at least one solvent (a).

In principle, various solvents can be used in the decolorizing agent, but the selection of specific solvents has proven to be particularly advantageous regarding achieving the best possible decolorizing effect.

Particularly suitable solvents include, for example, representatives from the group comprising benzyl alcohol, ethanol, phenoxyethanol, 2-phenylethanol, 1-pentanol, glycerol, 1,2-propylene glycol, 1,2-ethanediol, isopropanol, dipropylene glycol, N-octylpyrrolidone, methoxybutanol, ethyldiglycol, polyethylene glycol, 1,3-butanediol, 1,6-hexanediol, propylene carbonate and N,N-dimethyl-9-decenamide.

In the context of a further preferred embodiment, the decolorizing agent comprises at least one solvent (a) from the group comprising benzyl alcohol, ethanol, phenoxyethanol, 2-phenylethanol, 1-pentanol, glycerol, 1,2-propylene glycol, 1,2-ethanediol, isopropanol, dipropylene glycol, N-octylpyrrolidone, methoxybutanol, ethyl diglycol, polyethylene glycol, 1,3-butanediol, 1,6-hexanediol, propylene carbonate and N,N-dimethyl-9-decenamide.

Benzyl alcohol is also known as phenylmethanol and has the CAS number 100-51-6.

Ethanol has the Cas number 64-17-5.
Phenoxyethanol has the Cas number 122-99-6.
2-Phenylethanol is alternatively known as 2-phenylethyl alcohol and carries the CAS number 60-12-8.
Alternative names for 1-pentanol are pentan-1-ol, n-pentanol or amyl alcohol. 1-Pentanol has the CAS number 71-41-0.
Glycerol is also known alternatively as 1,2,3-propanetriol and has the CAS number 56-81-5.

1,2-Propylene glycol is alternatively referred to as 1,2-propanediol and has CAS numbers 57-55-6 [(RS)-1,2-dihydroxypropane], 4254-14-2 [(R)-1,2-dihydroxypropane], and 4254-15-3 [(S)-1,2-dihydroxypropane].

Ethylene glycol is also known alternatively as 1,2-ethanediol and has the CAS number 107-21-1.

Isopropanol is also known alternatively as 2-propanol and has the CAS number 67-63-0.

The dipropylene glycols (or oxydipropanyls) form a group of substances derived from the glycol ether. The technical product of the same name (in the singular) is a mixture of three structural isomers. A dipropylene glycol as contemplated herein is understood to be 2,2′-oxydi-1-propanol (CAS number 108-61-2), 1,1′-oxydi-2-propanol (CAS number 110-98-5) and 2-(2-hydroxypropoxy)-1-propanol (CAS number 106-62-7). The use of one of these dipropylene glycols in the decolorizing agent is encompassed by the present disclosure, as is the use of a mixture of second or all three of the structural isomers.

N-octylpyrrolidone is alternatively known as N-octyl-2-pyrrolidone or caprilyl-pyrrolidone and carries the CAS number 2687-94-7. This solvent can be obtained commercially, for example, under the trade name Surfadone LP 100 from the company Ashland (formerly ISP Global).

Methoxybutanol may alternatively be referred to as 3-methoxy-1-butanol and has the CAS number 2517-43-3. The solvent can be purchased, for example, as methoxybutanol from the Biesterfeld company. The IUPAC name for ethyldiglycol is 2-(2-ethoxyethoxy)-ethanol, ethyldiglycol has CAS number 111-90-0.

Polyethylene glycols in the sense of the present disclosure are polymers that are liquid at room temperature (25° C.) and have the general molecular formula C₂ₙH₄ₙ₊₂Oₙ₊₁. The repeating unit of the linear polymer is (—CH₂—CH₂—O—), with a molar mass of about 44 g mol-Chemically, it is a polyether. Particularly well-suited polyethylene glycols are the representatives with an average molecular mass between 200 g/mol and 400 g/mol, which are non-volatile liquids at room temperature.

1,3-Butanediol is alternatively referred to as butane-1,3-diol or 1,3-butylene glycol and has CAS numbers 107-88-0 (racemate), 6290-03-5 [(R)-1,3-butanediol] and 24621-61-2 [(S)-(+)-1,3-butanediol]. All stereoisomers of 1,3-butanediol are encompassed by the present disclosure.

An alternative name for 1,6-hexanediol is 1,6-dihydroxyhexane. 1,6-Hexanediol has the CAS number 629-11-8.

Propylene carbonate is alternatively known as 4-methyl-1,3-dioxolan-2-one or as propylene glycol carbonate or as carbonic acid propylene glycol ester and bears the CAS numbers 108-32-7 [(RS)-4-methyl-1,3-dioxolan-2-one], 51260-39-0 [(S)-4-methyl-1,3-dioxolan-2-one] and 16606-55-6 [(R)-4-methyl-1,3-dioxolan-2-one]. All stereoisomers of propylene carbonate are encompassed by the present disclosure.

N,N-dimethyl-9-decenamide has the CAS number 1356964-77-6.

In the context of a further particularly preferred embodiment, the decolorizing agent comprises at least one solvent (a) from the group comprising benzyl alcohol, ethanol, phenoxyethanol, 2-phenylethanol, 1-pentanol, glycerol, 1,2-propylene glycol, 1,2-ethanediol, isopropanol, dipropylene glycol, N-octylpyrrolidone, methoxybutanol, ethyl diglycol, 1,3-butanediol, 1,6-hexanediol and propylene carbonate.

Within the group of solvents mentioned above (a), the best results were obtained when benzyl alcohol was used as a solvent.

In the context of a further embodiment, explicitly quite particularly preferred is a process for decolorizing keratin material which has been colored by application of at least one organosilicon compound and at least one pigment, wherein a decolorizing agent comprising

(a) benzyl alcohol and

(b) at least one alkalizing agent

is applied to the dyed keratin material and rinsed off again after a contact time.

It has been found advantageous to use the above solvents (a) in certain ranges of amounts in the decolorizing agent. Particularly complete color removal could be observed if the decolorizing agent comprises—based on its total weight—one or more solvents (a) in a total amount of from 3 to 95% by weight, preferably from 5 to 75% by weight, more preferably from 6 to 55% by weight, still more preferably from 7 to 35% by weight and very particularly preferably from 8 to 15% by weight.

In the context of a further particularly preferred embodiment, the decolorizing agent comprises—based on the total weight of the decolorizing agent—one or more solvents (a) in a total amount of from 3 to 95% by weight, preferably from 5 to 75% by weight, more preferably from 6 to 55% by weight, still more preferably from 7 to 35% by weight and very particularly preferably from 8 to 15% by weight.

Benzyl alcohol, which is the solvent (a) with which the best decolorizing results were obtained, is also preferably used in the decolorizing agent in certain quantity ranges. Particularly satisfactory results were obtained when the decolorizing agent as contemplated herein comprises—based on its total weight—from 3 to 95% by weight, preferably from 5 to 75% by weight, more preferably from 6 to 55% by weight, still more preferably from 7 to 35% by weight and very particularly preferably from 8 to 15% by weight of benzyl alcohol.

In the context of a further particularly preferred embodiment, the decolorizing agent comprises—based on the total weight of the decolorizing agent—from 3 to 95% by weight, preferably from 5 to 75% by weight, more preferably from 6 to 55% by weight, still more preferably from 7 to 35% by weight and very particularly preferably from 8 to 15% by weight of benzyl alcohol.

### Alkalizing Agent (b)

As a second constituent essential to the invention, the decolorizing agent used in the process as contemplated herein comprises at least one alkalizing agent (b).

Suitable alkalizing agents can be selected from the group of C₁-C₆ alkanolamines, basic amino acids, ammonia, alkali metal metasilicates, alkaline earth metal metasilicates, alkali metal silicates, alkaline earth metal silicates, alkali metal hydroxides, alkaline earth metal hydroxides, alkali carbonates, alkaline earth carbonates, alkali hydrogen carbonates, alkali metal phosphates and alkaline earth metal phosphates.

Alkanolamines may be selected from primary amines having a C₂-C₆ alkyl parent bearing at least one hydroxyl group. Preferred alkanolamines are selected from the group formed by 2-aminoethan-1-ol (monoethanolamine), triethanolamine (alternatively referred to as tris(2-hydroxyethyl)amine), 3-aminopropan-1-ol, 4-aminobutan-1-ol, 5-aminopentan-1-ol, 1-aminopropan-2-ol, 1-aminobutan-2-ol, 1-aminopentan-2-ol, 1-aminopentan-3-ol, 1-aminopentan-4-ol, 3-amino-2-methylpropan-1-ol, 1-amino-2-methylpropan-2-ol, 3-aminopropane-1,2-diol, 2-amino-2-methylpropane-1,3-diol.

For the purposes of the present disclosure, an amino acid is an organic compound comprising in its structure at least one protonatable amino group and at least one —COOH or one —SO3H group. Preferred amino acids are aminocarboxylic acids, especially α-(alpha)-aminocarboxylic acids and ω-aminocarboxylic acids, whereby α-aminocarboxylic acids are particularly preferred.

As contemplated herein, basic amino acids are those amino acids which have an isoelectric point pI of greater than 7.0.

Basic α-aminocarboxylic acids contain at least one asymmetric carbon atom. In the context of the present disclosure, both enantiomers can be used equally as specific compounds or their mixtures, especially as racemates. However, it is particularly advantageous to use the naturally preferred isomeric form, usually in L-configuration.

The basic amino acids are preferably selected from the group formed by arginine, lysine, ornithine and histidine, especially preferably arginine and lysine. In another particularly preferred embodiment, the alkalizing agent is a basic amino acid from the group arginine, lysine, ornithine and/or histidine.

Ammonia can be used, for example, in the form of a 10 to 40% aqueous solution in the decolorizing agent.

Suitable alkali metal metasilicates include sodium metasilicate and potassium metasilicate. Suitable alkaline earth metal metasilicates include magnesium metasilicate and calcium metasilicate.

Suitable alkali metal silicates include sodium silicate and potassium silicate. Suitable alkaline earth metal metasilicates include magnesium silicate and calcium silicate.

Other inorganic alkalizing agents from the group of alkali metal hydroxides and alkaline earth metal hydroxides that can be used as contemplated herein include sodium hydroxide, potassium hydroxide, magnesium hydroxide, calcium hydroxide and barium hydroxide.

Other inorganic alkalizing agents from the group of alkali carbonates, alkaline earth carbonates and alkali hydrogen carbonates that can be used as contemplated herein are, for example, sodium carbonate, potassium carbonate, magnesium carbonate, calcium carbonate, sodium hydrogen carbonate and potassium hydrogen carbonate.

Other inorganic alkalizing agents from the group of alkali metal phosphates and alkaline earth metal phosphates that can be used as contemplated herein include sodium phosphate and potassium phosphate.

In another particularly preferred embodiment, the decolorizing agent comprises at least one alkalizing agent (b) selected from the group of C₁-C₆-alkanolamines, basic amino acids, ammonia, alkali metal metasilicates, alkaline earth metal metasilicates, alkali metal silicates, alkaline earth metal silicates, alkali metal hydroxides, alkaline earth metal hydroxides, alkali carbonates, alkaline earth carbonates, alkali hydrogen carbonates, alkali metal phosphates and alkaline earth metal phosphates.

A particularly good decolorizing effect could be achieved if the decolorizing agent used in the process as contemplated herein included 2-aminoethan-1-ol (monoethanolamine) and/or triethanolamine (alternatively also known as tris(2-hydroxyethyl)amine).

In the context of a further particularly preferred embodiment, the decolorizing agent comprises 2-aminoethan-1-ol and/or triethanolamine.

In the context of a further particularly preferred embodiment, the decolorizing agent comprises 2-aminoethan-1-ol and triethanolamine.

Furthermore, particularly satisfactory results in decolorization tests could be obtained if the decolorizing agent used in the process as contemplated herein included sodium metasilicate, potassium metasilicate, sodium silicate and/or potassium silicate.

In another particularly preferred embodiment, the decolorizing agent (b) comprises sodium metasilicate, potassium metasilicate, sodium silicate and/or potassium silicate.

### Water Content in Decolorizing Agent

To further optimize the decolorizing effect, it has proved advantageous to adjust the water content in the decolorizing agent to certain values. It is particularly preferred if the decolorizing agent comprises—based on the total weight of the decolorizing agent—from 5 to 70% by weight, preferably from 10 to 60% by weight, more preferably from 15 to 50% and very particularly preferably from 20 to 40% by weight of water.

In another particularly preferred embodiment, the decolorizing agent comprises—based on the total weight of the decolorizing agent—from 5 to 70% by weight, preferably from 10 to 60% by weight, further preferably from 15 to 50% by weight and very particularly preferably from 20 to 40% by weight of water. pH values of the decolorizing agent

In further trials, it was found that the decolorizing performance could be further improved by adjusting the optimum pH value. The best results were obtained when the decolorizing agent was adjusted to pH values in the range from 9.0 to 12.5, preferably from 9.5 to 12.0, more preferably from 9.5 to 11.5, and most preferably from 9.5 to 11.0.

In the context of a further particularly preferred embodiment, the decolorizing agent has a pH of from 9.0 to 12.5, preferably from 9.5 to 12.0, more preferably from 9.5 to 11.5, and most preferably from 9.5 to 11.0.

The pH values can be measured using the usual methods known from the prior art, such as measurement by employing glass electrodes via combination electrodes or via pH indicator paper. The pH values for the purposes of the present disclosure are pH values measured at a temperature of 22° C.

The pH value is preferably adjusted by using the alkalizing agent(s) (b) in the appropriate quantity ranges. To fine-tune the pH value, it may also be necessary to add acidifying agents in small quantities.

### Silicone Oils in Decolorizing Agent

To further improve the decolorizing effect, it has proved particularly advantageous to add at least one silicone oil to the decolorizing agent as a further ingredient.

In the context of a further particularly preferred embodiment, the decolorizing agent additionally comprises at least one silicone oil.

For the purposes of the present disclosure, the term “oil” is understood to mean a substance which is liquid at room temperature (25° C.). Furthermore, an oil as contemplated herein has a solubility in water of less than 1 g/l, preferably less than 0.5 g/l, more preferably less than 0.1 g/l (measured at 25° C.).

The water solubility of the silicone oil can be determined, for example, in the following way: 1.0 g of the silicone oil is added to a beaker. Then 1000 ml (1 liter) of water is added. A stir-fish is added, and the mixture is heated to 25° C. on a magnetic stirrer while stirring. It is stirred for 60 minutes. The aqueous mixture is then visually assessed. If a second phase is still visible after this period, i.e., a separately present oil phase in addition to the water phase, then the solubility of the silicone oil is less than 1 g/l (1 gram/liter).

The silicone oils optionally included in the decolorizing agent are polymeric compounds whose molecular weight is at least 500 g/mol, preferably at least 1000 g/mol, further preferably at least 2500 g/mol, and particularly preferably of at least 5000 g/mol.

These silicone oils comprise Si—O repeating units, where the Si atoms may carry organic radicals such as alkyl groups or substituted alkyl groups.

Corresponding to the high molecular weight of silicone oils, these are based on more than 10 Si—O repeating units, preferably more than 50 Si—O repeating units and particularly preferably more than 100 Si—O repeating units.

Silicone oils with a viscosity of 5 to 3000 mm²/s (measured according to ASTM standard D-445) have proved to be particularly suitable for solving this problem (measured at 25° C.).

It has been found to be particularly preferred to use in the decolorizing agent as a further optional constituent at least one silicone oil having a viscosity of from 5 to 3000 mm²/s, preferably from 10 to 2000 mm²/s, further preferably from 10 to 1000 mm²/s, still further preferably from 10 to 500 mm²/s and very particularly preferably from 10 to 500 mm²/s (always measured according to ASTM Standard D-445, 25° C.).

In the context of a further explicitly quite particularly preferred embodiment, the decolorizing agent additionally comprises at least one silicone oil having a viscosity of from 5 to 3000 mm²/s, preferably from 10 to 2000 mm²/s, further preferably from 10 to 1000 mm²/s, and particularly preferably from 10 to 500 mm²/s, measured according to ASTM Standard D-445.

The ASTM Standard D-445 is the standard method for measuring the kinematic viscosity of transparent and opaque liquids.

Viscosity was measured according to ASTM Standard D-446, Version 06 (D445-06), published June 2006. This measurement method measures the time required for the defined volume of a liquid to flow through the kappilars of a calibrated viscometer under defined conditions. For details of the procedure, please refer to ASMT-D445, ASTM D445-06. Measurement temperature is 25° C. Suitable equipment (such as viscometers and thermometers and the corresponding calibrations) are given in the method.

In principle, various silicone oils can be used in the decolorizing agent, but the use of polydimethylsiloxanes has proved to be particularly advantageous in terms of improving decolorizing performance.

For this reason, it is particularly preferred if the agent (c) comprises at least one silicone oil from the group of polydimethylsiloxanes (dimethicones).

In a further explicitly quite particularly preferred embodiment, the decolorizing agent comprises at least one silicone oil from the group of polydimethylsiloxanes.

Silicone oils from the group of linear polydimethylsiloxanes (PDMS) are compounds of the general structure (PDMS-I)

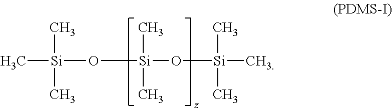

Here, z is selected so that the dimethicones are liquid and preferably have the very particularly suitable viscosity ranges.

Preferably, z can stand for an integer from 50 to 100000, more preferably from 100 to 50000, most preferably from 500 to 50000.

Corresponding dimethicones can be purchased commercially from various manufacturers. Particularly suitable, for example, is the dimethicone available for sale under the trade name Xiameter PMX 200 Silicone Fluid 50 CS from Dow Chemicals, which has a viscosity of 50 mm²/s (at 25° C.). This dimethicone is the most preferred.

Another particularly well-suited dimethicone is Xiameter PMX 200 Silicone Fluid 100 CS, also available from Dow Corning, which has a viscosity of 100 mm²/s (measured at 25° C.).

Another dimethicone that is particularly well-suited to this application is the Xiameter PMX 200 Silicone Fluid 350 CS, whose viscosity is 350 mm²/s (at 25° C.).

Another particularly well-suited dimethicone is Dow Corning 200 fluid 500 cSt, available from Dow Corning, which has a viscosity of 500 mm²/s (at 25° C.). The silicone oil(s) are preferably present in the decolorizing agent in certain quantity ranges. Very preferably, the decolorizing agent comprises—based on the total weight of the decolorizing agent—one or more silicone oils in a total amount of from 10 to 70% by weight, preferably from 15 to 60% by weight, further preferably from 20 to 50% by weight and very particularly preferably from 25 to 45% by weight.

In the context of a further preferred embodiment, the decolorizing agent comprises—based on the total weight of the decolorizing agent—one or more silicone oils in a total amount of from 10 to 70% by weight, preferably from 15 to 60% by weight, more preferably from 20 to 50% by weight and very particularly preferably from 25 to 45% by weight.

In a further preferred embodiment, the decolorizing agent comprises—based on the total weight of the decolorizing agent—one or more silicone oils from the group of polydimethylsiloxanes in a total amount of from 10 to 70% by weight, preferably from 15 to 60% by weight, more preferably from 20 to 50% by weight and very particularly preferably from 25 to 45% by weight.

### Surfactants in the Decolorizing Agent

The decolorizing performance of the decolorizing agent used in the process as contemplated herein can be further improved using at least one surfactant.

The term surfactants (T) refer to surface-active substances that can form adsorption layers on surfaces and interfaces or aggregate in bulk phases to form micelle colloids or lyotropic mesophases. A distinction is made between anionic surfactants comprising a hydrophobic residue and a negatively charged hydrophilic head group, amphoteric surfactants, which carry both a negative and a compensating positive charge, cationic surfactants, which in addition to a hydrophobic residue have a positively charged hydrophilic group, and non-ionic surfactants, which have no charges but strong dipole moments and are strongly hydrated in aqueous solution.

A particularly beneficial effect was observed if the decolorizing agent included at least one anionic surfactant.

In a very particularly preferred embodiment, the decolorizing agent comprises at least anionic surfactant.

Anionic surfactants as contemplated herein are exemplified by the presence of a water-solubilizing anionic group such as a carboxylate, sulfate, sulfonate or phosphate group and a lipophilic alkyl group having about 8 to 30 carbon atoms. In addition, glycol or polyglycol ether groups, ester, ether and amide and hydroxyl groups may also be present in the molecule.

Typical examples of anionic surfactants are alkyl benzene sulfonates, alkane sulfonates, olefin sulfonates, alkyl ether sulfonates, glycerol ether sulfonates, α-methyl ester sulfonates, sulfo fatty acids, Alkyl sulfates, fatty alcohol ether sulfates, glycerol ether sulfates, hydroxy mixed ether sulfates, monoglyceride (ether) sulfates, fatty acid amide (ether) sulfates, mono- and dialkyl sulfosuccinates, mono- and dialkylsulfosuccinamates, sulfotriglycerides, amide soaps, ether carboxylic acids and their salts, fatty acid isethionates, fatty acid sarcosinates, fatty acid taurides, acyl lactylates, acyl tartrates, acyl glutamates, acyl partates, alkyl oligoglucoside sulfates, protein fatty acid condensates (especially wheat-based vegetable products) and alkyl (ether) phosphates. If the anionic surfactants comprise polyglycol ether chains, they may have a conventional, but preferably a narrowed homologue distribution.

Examples of anionic surfactants as contemplated herein are, in each case in the form of the sodium, potassium and ammonium as well as the mono-, di- and trialkanolammonium salts with 2 to 4 C atoms in the alkanol group,
linear and branched fatty acids with 8 to 30 C-atoms (soaps),
Ether carboxylic acids of the formula R—O—(CH₂—CH₂O)ₓ—CH₂—COOH, in which R is a linear alkyl group having 8 to 30 carbon atoms and x=0 or 1 to 16,
Acyl sarcosides with 8 to 24 C atoms in the acyl group,
Acyltaurides with 8 to 24 C atoms in the acyl group,
Acyl isethionates with 8 to 24 C atoms in the acyl group, which are accessible by esterification of fatty acids with the sodium salt of 2-hydroxyethane sulfonic acid (isethionic acid). If fatty acids with 8 to 24 carbon atoms, e.g., lauric, myristic, palimitic or stearic acid, or also technical fatty acid fractions, e.g., the C₁₂-C₁₈ fatty acid fraction obtainable from coconut fatty acid, are used for this esterification, the C₁₂-C₁₈ acyl isethionates preferred as contemplated herein are obtained,
Sulfosuccinic acid mono- and dialkyl esters with 8 to 24 C atoms in the alkyl group and sulfosuccinic acid mono-alkyl polyoxyethyl esters with 8 to 24 C atoms in the alkyl group and 1 to 6 oxyethyl groups. Sulfosuccinic acid mono- and dialkyl esters can be prepared by reacting maleic anhydride with a fatty alcohol having 8-24 C atoms to form the maleic acid monoester of the fatty alcohol and further reacting with sodium sulfite to form the sulfosuccinic acid ester. Particularly suitable sulfosuccinic acid esters are derived from fatty alcohol fractions with 12-18 C atoms, such as those accessible from coconut fatty acid or coconut fatty acid methyl ester by hydrogenation,
linear alkane sulfonates with 8 to 24 C atoms,
linear alpha-olefin sulphonates with 8 to 24 C atoms,
Alpha-sulfofatty acid methyl esters of fatty acids with 8 to 30 C atoms,
Alkyl sulfates and alkyl polyglycol ether sulfates of the formula R—O(CH₂—CH₂O)ₓ—OSO₃H, in which R is a preferably linear alkyl group with 8 to 30 C atoms and x=0 or 1 to 12,
Hydroxysulfonates corresponding to at least one of the two following formulae or mixtures thereof, and salts thereof, their mixtures as well as their salt, CH₃—(CH₂)_{y}—CHOH—(CH₂)ₚ—(CH—SO₃M)-(CH₂)₂—CH₂—O—(CₙH₂ₙO)ₓ—H, and/or CH₃—(CH₂)_{y}—(CH—SO₃M)-(CH₂)ₚ—CHOH—(CH₂)₂—CH₂—O—(CₙH₂ₙO)ₓ—H where, in both formulae, y and z=0 or integers from 1 to 18, p=0, 1 or 2 and the sum (y+z+p) is a number from 12 to 18, x=0 or a number from 1 to 30 and n is an integer from 2 to 4 and M=H or alkali metal, in particular sodium, potassium, lithium, alkaline earth metal, in particular magnesium, calcium, zinc and/or an ammonium ion which may optionally be substituted, in particular mono-, di-, tri- or tetraammonium ions having C1 to C4 alkyl, alkenyl or aryl radicals,
sulfated hydroxyalkyl polyethylene and/or hydroxyalkylene propylene glycol ethers corresponding to the formula R¹—(CHOSO₃M)-CHR³—(OCHR⁴—CH₂)ₙ—OR₂ in which R¹ is a linear alkyl radical comprising 1 to 24 carbon atoms, R² is a linear or branched, saturated alkyl radical comprising 1 to 24 carbon atoms, R³ is hydrogen or a linear alkyl radical comprising 1 to 24 carbon atoms R⁴ is hydrogen or a methyl radical and M is hydrogen, ammonium, alkylammonium, alkanolammonium, in which the alkyl and alkanol radicals each have 1 to 4 carbon atoms, or a metal atom selected from lithium, sodium, potassium, calcium or magnesium, and n is a number in the range from 0 to 12, and furthermore the total number of carbon atoms included in R₁ and R³ is 2 to 44,
Sulfonates of unsaturated fatty acids with 8 to 24 C-atoms and 1 to 6 double bonds,
Esters of tartaric acid and citric acid with alcohols, which are addition products of about 2-15 molecules of ethylene oxide and/or propylene oxide to fatty alcohols with 8 to 22 C atoms,
Alkyl and/or alkenyl ether phosphates of the formula, R¹(OCH₂CH₂)ₙ—O—(PO—OX)—OR², where R¹ is preferably an aliphatic hydrocarbon radical of 8 to 30 carbon atoms, R² is hydrogen, a radical (CH₂CH₂O)ₙR² or X, n is from 1 to 10 and X is hydrogen, an alkali metal or alkaline earth metal or NR³R₄R⁵R₆, where R³ to R⁶ are each independently of the other's hydrogen or a C₁ to C₄-hydrocarbon radical,
sulfated fatty acid alkylene glycol esters of the formula RCO(AlkO)ₙSO₃M where RCO— is a linear or branched, aliphatic, saturated and/or unsaturated acyl radical having 6 to 22 carbon atoms, Alk is CH₂CH₂, CHCH₃CH₂ and/or CH₂CHCH₃, n is a number from 0.5 to 5 and M is a metal, such as alkali metal in particular sodium, potassium, lithium, alkaline earth metal, in particular magnesium, calcium, zinc, or ammonium ion, such as +NR³R₄R⁵R₆, where R³ to R₆ independently of one another represent hydrogen or a C1 to C4 hydrocarbon radical,
Monoglyceride sulfates and monoglyceride ether sulfates of the formula R⁸OC—(OCH₂CH₂)ₓ—OCH₂—[CHO(CH₂CH₂O)_{y}H]—CH₂O(CH₂CH₂O)₂—SO₃X, in which R⁸CO is a linear or branched acyl radical comprising 6 to 22 carbon atoms, x, y and z together represent 0 or numbers of 1 to 30, preferably 2 to 10, and X is an alkali metal or alkaline earth metal. Typical examples of monoglyceride (ether) sulphates suitable as contemplated herein are the reaction products of lauric acid monoglyceride, coco fatty acid monoglyceride, palmitic acid monoglyceride, stearic acid monoglyceride, oleic acid monoglyceride and tallow fatty acid monoglyceride as well as their ethylene oxide adducts with sulfur trioxide or chlorosulphonic acid in the form of their sodium salts. Preferably, monoglyceride sulfates are used in which RCO represents a linear acyl radical with 8 to 18 carbon atoms,
Amide ether carboxylic acids, R¹—CO—NR²—CH₂CH₂—O—(CH₂CH₂O)ₙCH₂COOM, where R¹ is a straight-chain or branched alkyl or alkenyl radical having a number of carbon atoms in the chain of from 2 to 30, n is an integer from 1 to 20 and R² is hydrogen, a methyl, ethyl, propyl, isopropyl, n-butyl, t-butyl or iso-butyl radical and M is hydrogen or a metal such as alkali metal, in particular sodium, potassium, lithium, alkaline earth metal, in particular magnesium, calcium, zinc, or an ammonium ion, such as ⁺NR³R⁴R⁵R⁶, where R³ to R⁶ independently of one another are hydrogen or a C1 to C4 hydrocarbon radical. Such products are available, for example, from the Chem-Y company under the product name Akypo®, and
An acylglutamate of the formula XOOC—CH₂CH₂CH(C(NH)OR)—COOX, in which RCO is a linear or branched acyl radical having 6 to 22 carbon atoms and 0 and/or 1, 2 or 3 double bonds and X is hydrogen, an alkali metal and/or alkaline earth metal, ammonium, alkylammonium, alkanolammonium or glucammonium.

Treatment of the dyed keratinous material with a decolorizing agent comprising the above-mentioned anionic surfactants gave particularly satisfactory results in decolorization tests.

Surprisingly, it was found that the decolorizing capacity could be further optimized by using one or more special anionic surfactants. A particularly good color fade could be obtained when the keratin material was treated with at least one anionic surfactant (b) from the group of the
Alkyl sulfates and alkyl polyglycol ether sulfates of the formula R—O(CH₂—CH₂O)ₓ—OSO₃H, in which R is a preferably linear alkyl group with 8 to 30 C atoms and x=0 or 1 to 12.

In a particularly preferred embodiment, decolorizing agent comprises at least one anionic surfactant selected from the group of the
Alkyl sulfates and alkyl polyglycol ether sulfates of the formula R—O(CH₂—CH₂O)ₓ—OSO₃H, in which R is a preferably linear alkyl group with 8 to 30 C atoms and x=0 or 1 to 12.

Preferably, the decolorizing agents as contemplated herein comprise one or more anionic surfactants in a total amount of from 1.0 to 20.0% by weight, preferably from 3.0 to 18.0% by weight, more preferably from 5.0 to 16.0% by weight and most preferably from 7.0 to 14.0% by weight. Here, the figures in % by weight is based on the total amount of all anionic surfactants, which are set in relation to the total amount of decolorizing agent.

In a further particularly preferred embodiment, the decolorizing agent comprises—based on the total weight of the agent—one or more anionic surfactants in a total amount of from 1.0 to 20.0% by weight, preferably from 3.0 to 18.0% by weight, more preferably from 5.0 to 16.0% by weight and very particularly preferably from 7.0 to 14.0% by weight.

### Fat Components in the Decolorizing Agent

As a further optional component, the decolorizing agent used in the process as contemplated herein may also comprise at least one fatty component.

The fatty components are hydrophobic substances that can form emulsions in the presence of water, forming micelle systems.

For the purposes of the present disclosure, “fatty components” means organic compounds with a solubility in water at room temperature (22° C.) and atmospheric pressure (760 mmHg) of less than 1% by weight, preferably less than 0.1% by weight. The definition of fat constituents explicitly covers only uncharged (i.e., non-ionic) compounds. Fat components have at least one saturated or unsaturated alkyl group with at least 12 C atoms. The molecular weight of the fat constituents is a maximum of 5000 g/mol, preferably a maximum of 2500 g/mol and particularly preferably a maximum of 1000 g/mol. The fat components are neither polyoxyalkylated nor polyglycerylated compounds.

Very preferably, the fat components are selected from the group of C₁₂-C₃₀ fatty alcohols, C₁₂-C₃₀ fatty acid triglycerides, C₁₂-C₃₀ fatty acid monoglycerides, C₁₂-C₃₀ fatty acid diglycerides and/or hydrocarbons.

In this context, very particularly preferred fat constituents are understood to be constituents from the group of C₁₂-C₃₀ fatty alcohols, C₁₂-C₃₀ fatty acid triglycerides, C₁₂-C₃₀ fatty acid monoglycerides, C₁₂-C₃₀ fatty acid diglycerides and/or hydrocarbons. For the purposes of the present disclosure, only non-ionic substances are explicitly regarded as fat components. Charged compounds such as fatty acids and their salts are not considered to be fat components.

The C₁₂-C₃₀ fatty alcohols can be saturated, mono- or polyunsaturated, linear or branched fatty alcohols with 12 to 30 C atoms.

Examples of preferred linear, saturated C₁₂-C₃₀ fatty alcohols are dodecan-1-ol (dodecyl alcohol, lauryl alcohol), tetradecan-1-ol (tetradecyl alcohol, myristyl alcohol), hexadecan-1-ol (hexadecyl alcohol, Cetyl alcohol, palmityl alcohol), octadecan-1-ol (octadecyl alcohol, stearyl alcohol), arachyl alcohol (eicosan-1-ol), heneicosyl alcohol (heneicosan-1-ol) and/or behenyl alcohol (docosan-1-ol).

Preferred linear unsaturated fatty alcohols are (9Z)-octadec-9-en-1-ol (oleyl alcohol), (9E)-octadec-9-en-1-ol (elaidyl alcohol), (9Z,12Z)-octadeca-9,12-dien-1-ol (linoleyl alcohol), (9Z,12Z,15Z)-octadeca-9,12,15-trien-1-ol (linolenoyl alcohol), gadoleyl alcohol ((9Z)-eicos-9-en-1-ol), arachidone alcohol ((5Z,8Z,11Z,14Z)-eicosa-5,8,11,14-tetraen-1-ol), erucyl alcohol ((13Z)-docos-13-en-1-ol) and/or brassidyl alcohol ((13E)-docosen-1-ol).

The preferred representatives for branched fatty alcohols are 2-octyl-dodecanol, 2-hexyl-dodecanol and/or 2-butyl-dodecanol.

In one embodiment, particularly good results were obtained when the decolorizing agent comprises one or more C₁₂-C₃₀ fatty alcohols selected from the group of dodecan-1-ol (dodecyl alcohol, lauryl alcohol), Tetradecan-1-ol (tetradecyl alcohol, myristyl alcohol), hexadecan-1-ol (hexadecyl alcohol, cetyl alcohol, palmityl alcohol), octadecan-1-ol (octadecyl alcohol, stearyl alcohol), arachyl alcohol (eicosan-1-ol), heneicosyl alcohol (heneicosan-1-ol), Behenyl alcohol (docosan-1-ol), (9Z)-octadec-9-en-1-ol (oleyl alcohol), (9E)-octadec-9-en-1-ol (elaidyl alcohol), (9Z,12Z)-octadeca-9,12-dien-1-ol (linoleyl alcohol), (9Z,12Z,15Z)-octadeca-9,12,15-trien-1-ol (linolenoyl alcohol), Gadoleyl alcohol ((9Z)-Eicos-9-en-1-ol), Arachidone alcohol ((5Z,8Z,11Z,14Z)-Eicosa-5,8,11,14-tetraen-1-ol), Erucyl alcohol ((13Z)-Docos-13-en-1-ol), Brassidyl alcohol ((13E)-docosen-1-ol) 2-octyl-dodecanol, 2-hexyl-dodecanol and/or 2-butyl-dodecanol.

In a very particularly preferred embodiment, the decolorizing agent comprises one or more C₁₂-C₃₀ fatty alcohols selected from the group of

Dodecan-1-ol (dodecyl alcohol, lauryl alcohol),
Tetradecan-1-ol (tetradecyl alcohol, myristyl alcohol),
Hexadecan-1-ol (hexadecyl alcohol, cetyl alcohol, palmityl alcohol),
Octadecan-1-ol (octadecyl alcohol, stearyl alcohol),
Arachyl alcohol (eicosan-1-ol),
Heneicosyl alcohol (heneicosan-1-ol),
Behenyl alcohol (docosan-1-ol),
(9Z)-Octadec-9-en-1-ol (oleyl alcohol),
(9E)-Octadec-9-en-1-ol (elaidyl alcohol),
(9Z,12Z)-Octadeca-9,12-dien-1-ol (linoleyl alcohol),
(9Z,12Z,15Z)-Octadeca-9,12,15-trien-1-ol (linolenoyl alcohol),
Gadoleyl alcohol ((9Z)-Eicos-9-en-1-ol),
Arachidonic alcohol ((5Z,8Z,11Z,14Z)-Eicosa-5,8,11,14-tetraen-1-ol),
Erucyl alcohol ((13Z)-docos-13-en-1-ol),
Brassidyl alcohol ((13E)-docosen-1-ol),

### 2-Octyl-dodecanol,

2-hexyl dodecanol and/or

### 2-Butyl-dodecanol.

It has been found to be quite preferable to use one or more C₁₂-C₃₀ fatty alcohols in quite specific ranges of amounts.

It is particularly preferred if the decolorizing agent comprises—based on the total weight of the decolorizing agent—one or more C₁₂-C₃₀ fatty alcohols in a total amount of from 2.0 to 50.0% by weight, preferably from 4.0 to 40.0% by weight, more preferably from 6.0 to 30.0% by weight, still more preferably from 8.0 to 20.0% by weight and most preferably from 10.0 to 15.0% by weight.

In another particularly preferred embodiment, the decolorizing agent comprises—based on the total weight of the decolorizing agent—one or more C₁₂-C₃₀ fatty alcohols in a total amount of from 2.0 to 50.0 wt. % by weight, preferably from 4.0 to 40.0% by weight, more preferably from 6.0 to 30.0% by weight, still more preferably from 8.0 to 20.0% by weight and very particularly preferably from 10.0 to 15.0% by weight.

Furthermore, as a very particularly preferred fat constituent, the decolorizing agent may also comprises at least one C₁₂-C₃₀ fatty acid triglyceride, the C₁₂-C₃₀ fatty acid monoglyceride and/or C₁₂-C₃₀ fatty acid diglyceride. For the purposes of the present disclosure, a C₁₂-C₃₀ fatty acid triglyceride is understood to be the triester of the trivalent alcohol glycerol with three equivalents of fatty acid. Both structurally identical and different fatty acids within a triglyceride molecule can be involved in the formation of esters.

As contemplated herein, fatty acids are to be understood as saturated or unsaturated, unbranched or branched, unsubstituted or substituted C₁₂-C₃₀ carboxylic acids. Unsaturated fatty acids can be mono- or polyunsaturated. For an unsaturated fatty acid, its C═C double bond(s) may have the Cis or Trans configuration.

Fatty acid triglycerides are particularly suitable in which at least one of the ester groups is formed from glycerol with a fatty acid selected from dodecanoic acid (lauric acid), tetradecanoic acid (myristic acid), hexadecanoic acid (palmitic acid), tetracosanoic acid (lignoceric acid), octadecanoic acid (stearic acid), eicosanoic acid (arachidic acid), docosanoic acid (behenic acid), petroselinic acid [(Z)-6-octadecenoic acid], palmitoleic acid [(9Z)-hexadec-9-enoic acid], oleic acid [(9Z)-octadec-9-enoic acid], elaidic acid [(9E)-octadec-9-enoic acid], erucic acid [(13Z)-docos-13-enoic acid], linoleic acid [(9Z,12Z)-octadeca-9,12-dienoic acid, linolenic acid [(9Z,12Z,15Z)-octadeca-9,12,15-trienoic acid, eleostearic acid [(9Z,11E,13E)-octadeca-9,11,3-trienoic acid], arachidonic acid [(5Z,8Z,11Z,14Z)-icosa-5,8,11,14-tetraenoic acid], and/or nervonic acid [(15Z)-tetracos-15-enoic acid].

The fatty acid triglycerides can also be of natural origin. The fatty acid triglycerides or mixtures thereof occurring in soybean oil, peanut oil, olive oil, sunflower oil, macadamia nut oil, moringa oil, apricot kernel oil, marula oil, avocado oil, almond oil and/or optionally hydrogenated castor oil are particularly suitable for use in the product as contemplated herein.

A C₁₂-C₃₀ fatty acid monoglyceride is understood to be the monoester of the trivalent alcohol glycerol with one equivalent of fatty acid. Either the middle hydroxy group of glycerol or the terminal hydroxy group of glycerol may be esterified with the fatty acid.

C₁₂-C₃₀ fatty acid monoglycerides are particularly suitable in which a hydroxyl group of glycerol is esterified with a fatty acid, the fatty acids being selected from dodecanoic acid (lauric acid), tetradecanoic acid (myristic acid), hexadecanoic acid (palmitic acid), tetracosanoic acid (lignoceric acid), octadecanoic acid (stearic acid), eicosanoic acid (arachidic acid), docosanoic acid (behenic acid), petroselinic acid [(Z)-6-octadecenoic acid], palmitoleic acid [(9Z)-hexadec-9-enoic acid], oleic acid [(9Z)-octadec-9-enoic acid], elaidic acid [(9E)-octadec-9-enoic acid], erucic acid [(13Z)-docos-13-enoic acid], linoleic acid [(9Z,12Z)-octadeca-9,12-dienoic acid, linolenic acid [(9Z,12Z,15Z)-octadeca-9,12,15-trienoic acid, eleostearic acid [(9Z,11E,13E)-octadeca-9,11,3-trienoic acid], arachidonic acid [(5Z,8Z,11Z,14Z)-icosa-5,8,11,14-tetraenoic acid], or nervonic acid [(15Z)-tetracos-15-enoic acid].

A C₁₂-C₃₀ fatty acid diglyceride is the diester of the trivalent alcohol glycerol with two equivalents of fatty acid. Either the middle and one terminal hydroxy group of glycerol may be esterified with two equivalents of fatty acid, or both terminal hydroxy groups of glycerol are esterified with one fatty acid each. The glycerol can be esterified with two structurally identical fatty acids or with two different fatty acids.

Fatty acid triglycerides are particularly suitable in which at least one of the ester groups is formed from glycerol with a fatty acid selected from dodecanoic acid (lauric acid), tetradecanoic acid (myristic acid), hexadecanoic acid (palmitic acid), tetracosanoic acid (lignoceric acid), octadecanoic acid (stearic acid), eicosanoic acid (arachidic acid), docosanoic acid (behenic acid), petroselinic acid [(Z)-6-octadecenoic acid], palmitoleic acid [(9Z)-hexadec-9-enoic acid], oleic acid [(9Z)-octadec-9-enoic acid], elaidic acid [(9E)-octadec-9-enoic acid], erucic acid [(13Z)-docos-13-enoic acid], linoleic acid [(9Z,12Z)-octadeca-9,12-dienoic acid, linolenic acid [(9Z,12Z,15Z)-octadeca-9,12,15-trienoic acid, eleostearic acid [(9Z,11E,13E)-octadeca-9,11,3-trienoic acid], arachidonic acid [(5Z,8Z,11Z,14Z)-icosa-5,8,11,14-tetraenoic acid], and/or nervonic acid [(15Z)-tetracos-15-enoic acid].

Particularly good results were obtained when composition (B) included at least one C₁₂-C₃₀ fatty acid monoglyceride selected from the monoesters of glycerol with one equivalent of fatty acid selected from the group of dodecanoic acid (lauric acid), Tetradecanoic acid (myristic acid), hexadecanoic acid (palmitic acid), tetracosanoic acid (lignoceric acid), octadecanoic acid (stearic acid), eicosanoic acid (arachidic acid), docosanoic acid (behenic acid), Petroselinic acid [(Z)-6-octadecenoic acid], palmitoleic acid [(9Z)-hexadec-9-enoic acid], oleic acid [(9Z)-octadec-9-enoic acid], elaidic acid [(9E)-octadec-9-enoic acid], erucic acid [(13Z)-docos-13-enoic acid], linoleic acid [(9Z,12Z)-octadeca-9,12-dienoic acid, linolenic acid [(9Z,12Z,15Z)-octadeca-9,12,15-trienoic acid, eleostearic acid [(9Z,11E,13E)-octadeca-9,11,3-trienoic acid], arachidonic acid [(5Z,8Z,11Z,14Z)-icosa-5,8,11,14-tetraenoic acid] and/or nervonic acid [(15Z)-tetracos-15-enoic acid].

In a particularly preferred embodiment, the second composition (B) comprises at least one C₁₂-C₃₀ fatty acid monoglyceride (B2) selected from the monoesters of glycerol with one equivalent of fatty acid selected from the group of dodecanoic acid, tetradecanoic acid, hexadecanoic acid, tetracosanoic acid, octadecanoic acid, eicosanoic acid and/or docosanoic acid.

The choice of suitable amounts of C₁₂-C₃₀ fatty acid mono-, C₁₂-C₃₀ fatty acid di- and/or C₁₂-C₃₀ fatty acid triglycerides can also have a particularly strong influence on the rate of film formation originating from the C₁-C₆ alkoxy silanes. For this reason, it has proved particularly preferable to use one or more C₁₂-C₃₀ fatty acid mono-, C₁₂-C₃₀ fatty acid di- and/or C₁₂-C₃₀ fatty acid triglycerides in specific ranges of amounts in the decolorizing agent.

With regard to the solution to the problem as contemplated herein, it has proven to be particularly preferred if the decolorizing agent—based on the total weight of the decolorizing agent—comprises one or more C₁₂-C₃₀ fatty acid mono-, C₁₂-C₃₀ fatty acid di- and/or C₁₂-C₃₀-fatty acid triglycerides in a total amount of 0.1 to 20.0% by weight, preferably 0.3 to 15.0% by weight, more preferably 0.5 to 10.0% by weight and very particularly preferably 0.8 to 5.0% by weight.

The C₁₂-C₃₀ fatty acid mono-, C₁₂-C₃₀ fatty acid di- and/or C₁₂-C₃₀ fatty acid triglycerides can be used as the sole fatty ingredients in the decolorizing agent. However, it is particularly preferred to incorporate at least one C₁₂-C₃₀ fatty acid mono-, C₁₂-C₃₀ fatty acid di- and/or C₁₂-C₃₀ fatty acid triglyceride in combination with at least one C₁₂-C₃₀ fatty alcohol into the decolorizing agent.

Furthermore, as a very particularly preferred fatty constituent, the decolorizing agent may also comprise at least one hydrocarbon.

Hydrocarbons are compounds comprising exclusively of the atoms carbon and hydrogen with 8 to 80 C atoms. In this context, aliphatic hydrocarbons such as mineral oils, liquid paraffin oils (e.g., Paraffinium Liquidum or Paraffinum Perliquidum), isoparaffin oils, semi-solid paraffin oils, paraffin waxes, hard paraffin (Paraffinum Solidum), vaseline and polydecenes are particularly preferred.

Liquid paraffin oils (Paraffinum Liquidum and Paraffinium Perliquidum) have proven to be particularly suitable in this context. Paraffinum Liquidum, also known as white oil, is the preferred hydrocarbon. Paraffinum Liquidum is a mixture of purified, saturated, aliphatic hydrocarbons, comprising hydrocarbon chains with a C-chain distribution of 25 to 35 C-atoms.

Particularly satisfactory results were obtained when the decolorizing agent included at least one hydrocarbon selected from the group of mineral oils, liquid kerosene oils, isoparaffin oils, semisolid kerosene oils, kerosene waxes, hard kerosene (Paraffinum solidum), petrolatum and polydecenes.

In a very particularly preferred embodiment, the decolorizing agent comprises at least one fatty constituent from the group of hydrocarbons.

It has also been found to be particularly preferred to use one or more hydrocarbons in specific ranges of amounts in the decolorizing agent.

Regarding the solution of the problem as contemplated herein, it proved to be quite particularly preferable if the decolorizing agent included—based on the total weight of the decolorizing agent—one or more hydrocarbons in a total amount of from 0.5 to 20.0% by weight, preferably from 1.0 to 15.0% by weight, more preferably from 1.5 to 10.0% by weight and most preferably from 2.0 to 8.0% by weight.

In a very particularly preferred embodiment, the decolorizing agent comprises—based on the total weight of the decolorizing agent—one or more hydrocarbons in a total amount of from 0.5 to 20.0% by weight, preferably from 1.0 to 15.0% by weight, more preferably from 1.5 to 10.0% by weight and very particularly preferably from 2.0 to 8.0% by weight.

### Other Cosmetic Ingredients in the Decolorizing Agent

In addition to the ingredients essential to the present disclosure and optionally usable as described above, the decolorizing agent may also contain one or more further cosmetic ingredients.

The selection of these other substances will be made by the specialist according to the desired properties of the agents. Regarding other optional components and the quantities of these components used, explicit reference is made to the relevant manuals known to the specialist.

### Application of the Decolorizing Agent

In the process as contemplated herein, the previously described decolorizing agent is applied to applied to the dyed keratin material and rinsed off again after a contact time.

The application can be done by hand or with the help of an applicator, such as a brush or an aplicette, or even a brush or a comb.

Depending on whether the user wants complete decolorization or only certain sections or areas. Strands are to be decolorized, the decolorizing agent can be applied either to the entire keratinous material (such as the entire previously colored scalp hair) or to specific parts or corresponding strands of the keratinous material or keratin fibers.

After application, the decolorizing agent is left to act on the keratin material for a certain period. For example, the exposure time may be from 5 to 60 minutes, preferably from 5 to 50 minutes, more preferably from 5 to 40 minutes, and most preferably from 5 to 30 minutes. After this exposure time, the decolorizing agent is rinsed out with water again

In a further preferred embodiment, the decolorizing agent is applied to the colored keratin material and rinsed off again after an exposure time of 5 to 60 minutes, preferably of 5 to 50 minutes, further preferably of 5 to 40 minutes and very particularly preferably of 5 to 30 minutes.

The decolorizing agent can be applied to the keratin material at room temperature or at body temperature. However, to support or accelerate color removal, the keratin material exposed to the decolorizing agent can also be exposed to elevated temperatures. It is as contemplated herein if the decolorizing agent is applied to the dyed keratin material and the keratin material is heated to a temperature of 25 to 70° C., preferably 25 to 60° C., more preferably 30 to 55° C. and very particularly preferably 40 to 55° C. during the action of the decolorizing agent.

In the context of a further embodiment,
the decolorizing agent is applied to the dyed keratin material
the keratin material is heated during the action of the decolorizing agent to a temperature of from 25 to 70°, preferably from 25 to 60° C., more preferably from 30 to 55° C. and most preferably from 40 to 55° C., and then
the decolorizing agent is rinsed off again.

In addition to providing thermal support for the decolorization process, it is also possible to subject the keratin material to which the decolorizing agent has been applied to mechanical stress to improve the detachment of the film formed on the keratin material during coloring. For example, the keratin material can be massaged with the hands or combed with a comb or brush during the decolorization process. Any other mechanical stress suitable for improving the detachment of the colored film from the keratin material under the action of the decolorizing agent is also conceivable and encompassed by the process as contemplated herein.

In the context of a further preferred embodiment,
the decolorizing agent is applied to the dyed keratin material,
the keratin material is combed, massaged, brushed or otherwise subjected to mechanical force during the action of the decolorizing agent, and then
the decolorizing agent is rinsed off again.

As previously described, the decolorizing agent as contemplated herein can be applied to decolorize keratin material that has been colored by applying at least one organosilicon compound and at least one pigment. If, for example, the user discovers after dyeing that the color result does not meet his requirements, he can take this as an opportunity to remove the dyeing again by applying the decolorizing agent.

Furthermore, the user can also plan a coloring and the subsequent decolorization from the outset, for example, if he wants to dye his hair for a particular occasion and then decolorize it again. For this purpose, the user can also be provided with all the agents or formulations necessary for both coloration and decolorization.

Thus, a second object of the present disclosure is a method for coloring and later decolorizing human hair, comprising the following steps:

(1) Applying a colorant to the hair, the colorant comprising one or more organic C₁-C₆ alkoxy silanes and/or condensation products thereof, and one or more pigments,
(2) Allow the dye to act on the hair,
(3) Rinse the dye from the hair,
(4) applying an aftertreatment agent to the hair, wherein the aftertreatment agent comprises at least one film-forming polymer,
(5) Allow the after-treatment agent to act on the hair,
(6) Rinse the after-treatment product out of the hair,
(7) Applying a decolorizing agent, as disclosed in detail in the description of the first subject present disclosure, to the hair,
(8) Allow the decolorizing agent to act on the hair and
(9) Rinse the decolorizing agent out of the hair.

Thus, another object of the present disclosure is to provide a method for coloring and later decolorizing human hair, comprising the following steps:

(1) Applying a pretreatment agent to the hair, wherein the pretreatment agent comprises one or more organic C₁-C₆ alkoxy silanes and/or condensation products thereof,
(2) Allow the pre-treatment agent to act on the hair,
(3) Rinse the pre-treatment agent out of the hair,
(4) Applying a colorant to the hair, wherein colorant comprises one or more pigments,
(5) Allow the dye to act on the hair,
(6) Rinse the dye from the hair,
(7) Applying to the hair a decolorizing agent as defined in any one of claims 1 to 11,
(8) Allow the decolorizing agent to act on the hair and
(9) Rinse the decolorizing agent from the hair.

The organic C₁-C₆ alkoxy silanes and/or their condensation products have already been disclosed in detail in the description of the first subject matter of the present disclosure. The pigments have already been disclosed in detail in the description of the first subject matter of the present disclosure. The decolorizing agent has also already been disclosed in detail in the description of the first subject matter of the present disclosure.

In yet another preferred embodiment, a corresponding method comprising the (4) Applying a colorant to the hair, wherein colorant comprises one or more pigments and one or more film-forming polymers.

The film-forming polymers have already been disclosed in detail in the description of the first subject matter of the present disclosure.

### Multi-Component Packaging Unit

It is particularly convenient for the user if the appropriate coloring and decolorizing agents are made available to him in the form of a multi-component packaging unit.

Thus, another object of the present disclosure is to provide a multi-component packaging unit (kit-of-parts) for coloring and decolorizing keratin material, comprising separately prepared:
a first container with an agent comprising one or more organic C₁-C₆ alkoxy silanes and/or their condensation products,
a second container with an agent comprising at least one pigment,
a third container comprising a decolorizing agent as disclosed in detail in the description of the first subject matter of the present disclosure, and
optionally a fourth container with an agent comprising at least one film-forming polymer.

Another object of the present disclosure is the use of a decolorizing agent, as disclosed in detail in the description of the first object of the present disclosure, for decolorizing keratinous material which has been colored by application of at least one organic C₁-C₆ alkoxy-silane and/or a condensation product thereof and by application of at least one pigment.

The organic C₁-C₆ alkoxy silanes and/or their condensation products have already been disclosed in detail in the description of the first subject matter of the present disclosure. The pigments have already been disclosed in detail in the description of the first subject matter of the present disclosure. The decolorizing agent has also already been disclosed in detail in the description of the first subject matter of the present disclosure.

Concerning the further preferred embodiments of the multicomponent packaging unit as contemplated herein and the use, mutatis mutantis what has been said about the methods as contemplated herein applies.

### EXAMPLES

### 1. Keratinous Material Staining

### 1.1. Preparation of the Silane Blend (Composition (A))

A reactor with a heatable/coolable outer shell and with a capacity of 10 liters was filled with 4.67 kg of methyltrimethoxysilane (34.283 mol). With stirring, 1.33 kg of (3-aminopropyl)triethoxysilane (6.008 mol) was then added. This mixture was stirred at 30° C. Subsequently, 670 ml of distilled water (37.18 mol) was added dropwise with vigorous stirring while maintaining the temperature of the reaction mixture at 30° C. under external cooling. After completion of the water addition, stirring was continued for another 10 minutes. A vacuum of 280 mbar was then applied, and the reaction mixture was heated to a temperature of 44° C. Once the reaction mixture reached the temperature of 44° C., the ethanol and methanol released during the reaction were distilled off over a period of 190 minutes. During distillation, the vacuum was lowered to 200 mbar. The distilled alcohols were collected in a chilled receiver. The reaction mixture was then allowed to cool to room temperature. To the mixture thus obtained, 3.33 kg of hexamethyldisiloxane was then dropped while stirring. It was stirred for 10 minutes. In each case, 100 ml of the silane blend was filled into a bottle with a capacity of 100 ml and screw cap closure with seal. After filling, the bottles were tightly closed. The water content was less than 2.0% by weight.

### 1.2. Preparation of the Composition (B)

The following compositions (B) were prepared (unless otherwise indicated, all figures are in wt. %).

Composition (B)

| | wt. % |
|---|---|
| Hydroxyethylcellulose | — |
| Cetyl alcohol (C₁₆ fatty alcohol) | 7.2 |
| Stearyl alcohol (C₁₈ fatty alcohol) | 4.0 |
| Paraffinum Liquidum | 4.2 |
| Ceteareth-30 (Cetearyl alcohol, ethoxylated 30 EO) | 2.4 |
| Brij S 100 PA SG (stearyl alcohol, ethoxylated 100 EO, | 1.2 |
| Croda) | |
| Cutina GMS V (INCI: Glyceryl stearate, glyceryl | 1.2 |
| mono/dipalmitate/stearate) CAS No. 85251-77-0 | |
| 1.2-propanediol | 12.0 |
| Water (dist.) | ad 100 |

### 1.3. Preparation of Compositions (C) and (D)

The following compositions were prepared (unless otherwise stated, all figures are in wt. %).

Composition (C)

| | wt. % |
|---|---|
| Lavanya Belmont | 35.0 |
| Phthalocyanine blue pigment CI 74160 | |
| PEG-12 Dimethicone | ad 100 |

Composition (D)

| | wt. % |
|---|---|
| Ethylene/Sodium Acrylate Copolymer (25% solution) | 40.0 |
| Water | ad 100 |

### 1.4. Application (Staining Step)

The ready-to-use composition was prepared by mixing 1.5 g of composition (A), 20.0 g of composition (B) and 1.5 g of composition (C), respectively. Compositions (A), (B) and (C) were shaken for 1 minute each, then this ready-to-use agent was dyed on hair strands (Kerling, Euronatural hair white).

Three minutes after completion of shaking, the ready-to-use composition was applied to the hair strands, left to act for 1 min, and then rinsed out.

Subsequently, the composition (D) was applied to each hair strand, left to act for 1 minute and then also rinsed with water. After that, the dyed strands were left to dry at room temperature. The strands were left to rest for 48 hours.

### 2. Decolorization of the Keratinic Material

The following decolorizing agents were prepared (unless otherwise stated, all figures are in % by weight).

| Decolorizing agent | (E1) | (E2) |
|---|---|---|
| Sodium laureth sulfate (sodium lauryl ether | 8.6 | 8.6 |
| sulfate) | | |
| CAS No. 68891-38-3 | | |
| Benzyl alcohol | 11.7 | 11.7 |
| Dimethicone 5 cST (25° C.) | 41.0 | — |
| (5 mm2/s, 25° C.) | | |
| Dimethicone 50 cST (25° C.) | — | 41.0 |
| (50 mm2/s, 25° C.) | | |
| 2-Aminoethan-1-ol | 0.8 | 0.8 |
| Triethanolamine | 2.5 | 2.5 |
| Water (dist.) | ad 100 | ad 100 |

The previously dyed strands were each treated with a decolorizing agent. After application, the decolorizer was left to act on the strands for 30 minutes at room temperature. After an exposure time of 5 minutes and after 15 minutes, the hair wetted with the decolorizing agent was massaged for 3 minutes each.

After completion of the reaction, the decolorizing agent was rinsed out with water. Then the strands were dried.

The dry strands were each visually evaluated under a daylight lamp after dyeing and after decolorization

| | | |
|---|---|---|
| Coloring, step 1: | (A) + (B) + (C) | (A) + (B) + (C) |
| Coloring, step 2: | (D) | (D) |
| Color result coloring | blue +++ | blue +++ |
| Decolorization | (E1) | (E2) |
| Color result decolorization | light blue + | light blue + |

Color intensity:
+++ = high
++ = medium
+ = low

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A process for decolorizing keratinous material which has been colored by the application of at least one organosilicon compound and at least one pigment, wherein the process comprises:
applying a decolorizing agent to the keratinous material, wherein the decolorizing agent comprises:
(a) at least one solvent, and
(b) at least one alkalizing agent; and
rinsing the decolorizing agent from the keratinous material after a contact time.

2. The process according to claim 1, wherein the decolorizing agent is applied to keratinous material which has been colored by application of at least one organic C₁-C₆ alkoxy-silane and/or a condensation product thereof and by application of at least one pigment.

3. The process according to claim 1, wherein the decolorizing agent is applied to keratinous material which has been colored by application of at least one organic C₁-C₆ alkoxy-silane and/or a condensation product thereof, by application of at least one pigment and by application of at least one film-forming polymer.

4. The process according to claim 1, wherein the decolorizing agent comprises at least one solvent (a) selected from the group consisting of benzyl alcohol, ethanol, phenoxyethanol, 2-phenylethanol, 1-pentanol, glycerol, 1,2-propylene glycol, 1,2-ethanediol, isopropanol, dipropylene glycol, N-octylpyrrolidone, methoxybutanol, ethyl diglycol, polyethylene glycol, 1,3-butanediol, 1,6-hexanediol, propylene carbonate or N,N-dimethyl-9-decenamide.

5. The process according to claim 1, wherein the decolorizing agent comprises—based on the total weight of the decolorizing agent—one or more solvents (a) in a total amount of from about 3 to about 95% by weight.

6. The process according to a claim 1, wherein the decolorizing agent comprises—based on the total weight of the decolorizing agent—from about 3 to about 95% by weight of benzyl alcohol.

7. The process according to claim 1, wherein the decolorizing agent comprises at least one alkalizing agent (b) selected from the group consisting of C₁-C₆-alkanolamines, basic amino acids, ammonia, alkali metal metasilicates, alkaline earth metal metasilicates, alkali metal silicates, alkaline earth metal silicates, alkali metal hydroxides, alkaline earth metal hydroxides, alkali carbonates, alkaline earth metal carbonates, alkali hydrogen carbonates, alkali metal phosphates or alkaline earth metal phosphates.

8. The process according to claim 1, wherein the decolorizing agent (b) comprises 2-aminoethan-1-ol and/or triethanolamine.

9. The process according to claim 1, wherein the decolorizing agent (b) comprises sodium metasilicate, potassium metasilicate, sodium silicate and/or potassium silicate.

10. The process according to claim 1, wherein the decolorizing agent comprises—based on the total weight of the decolorizing agent—from about 5 to about 70% by weight of water.

11. The process according to claim 1, wherein the decolorizing agent has a pH of from about 9.0 to about 12.5.

12. The process according to claim 1, wherein the decolorizing agent additionally comprises at least one silicone oil.

13. The process according to claim 12, wherein the decolorizing agent additionally comprises at least one silicone oil having a viscosity of from about 5 to about 3000 mm²/s, measured according to ASTM standard D-445.

14. (canceled)

15. The process according to claim 12, wherein the decolorizing agent comprises—based on the total weight of the decolorizing agent—one or more silicone oils in a total amount of from 10 to 70% by weight.

16. The process according to claim 1, wherein the decolorizing agent comprises at least one anionic surfactant.

17. The process according to claim 1, wherein the decolorizing agent is applied to the colored keratin material and rinsed off again after an exposure time of from about 5 to about 60 minutes.

18. (canceled)

19. The process according to claim 1, further comprising the following steps:
(1) Applying a colorant to the hair, the colorant comprising one or more organic C₁-C₆ alkoxy silanes and/or condensation products thereof, and one or more pigments,
(2) Allowing the dye to act on the hair,
(3) Rinsing the dye from the hair,
(4) Applying an aftertreatment agent to the hair, wherein the aftertreatment agent comprises at least one film-forming polymer,
(5) Allowing the after-treatment agent to act on the hair,
(6) Rinsing the after-treatment product out of the hair,
(7) Applying to the hair the decolorizing agent,
(8) Allowing the decolorizing agent to act on the hair and
(9) Rinsing the decolorizing agent from the hair.

20. The process according to claim 1, further comprising the following steps:
(1) Applying a pretreatment agent to the hair, wherein the pretreatment agent comprises one or more organic C₁-C₆ alkoxy silanes and/or condensation products thereof,
(2) Allow the pre-treatment agent to act on the hair,
(3) Rinsing the pre-treatment agent out of the hair,
(4) Applying a colorant to the hair, wherein colorant contains one or more pigments,
(5) Allowing the dye to act on the hair,
(6) Rinsing the dye from the hair,
(7) Applying to the hair the decolorizing agent,
(8) Allowing the decolorizing agent to act on the hair and
(9) Rinsing the decolorizing agent from the hair.

21. The process of claim 20,
(4) wherein applying the colorant to the hair comprises applying colorant comprising one or more pigments and one or more film-forming polymers.

22. Multi-component packaging unit for staining and decolorizing keratin material, comprising separately packaged:
a first container with an agent comprising one or more organic C₁-C₆ alkoxy silanes and/or their condensation products,
a second container with an agent comprising at least one pigment,
a third container with a decolorizing agent as defined in claim 1, and
optionally a fourth container with an agent comprising at least one film-forming polymer.

23. (canceled)
